(19) United States
(12) Patent Application Publication
(10) Pub. No.: US 2026/0253402 A1
(43) Pub. Date: Aug. 27, 2026

(54) SYSTEM

(71) Applicant: SoftBank Group Corp., Tokyo (JP)

(72) Inventor: Hironobu HOKI, Tokyo (JP)

(73) Assignee: SoftBank Group Corp., Tokyo (JP)

(21) Appl. No.: 19/537,613

(22) Filed: Feb. 12, 2026

(30) Foreign Application Priority Data
Feb. 21, 2025 (JP) 2025-026997

(51) Int. Cl. G06V 10/82; G06N 3/045; G06V 10/764; G06V 10/94; G10L 25/30; G16H 20/00; G16H 30/40; G16H 50/30

(52) U.S. Cl. CPC G06V 10/82; G06N 3/045; G06V 10/764; G06V 10/95; G10L 25/30; G16H 20/00; G16H 30/40; G16H 50/30

(57) ABSTRACT

The system according to the embodiment comprises an acquisition unit, an analysis unit, and a proposal unit. The acquisition unit acquires an image of an emergency patient. The analysis unit analyzes the image acquired by the acquisition unit. The proposal unit proposes a product based on a diagnosis result obtained by the analysis unit.

### CROSS-REFERENCE TO RELATED APPLICATION

The present application claims priority to and incorporates by reference the entire contents of Japanese Patent Application No. 2025-026997 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, rapid diagnosis and appropriate product proposals for emergency patients have not been sufficiently performed, and there is room for improvement.

### SUMMARY OF THE INVENTION

The system according to the embodiment comprises an acquisition unit, an analysis unit, and a proposal unit. The acquisition unit acquires an image of an emergency patient. The analysis unit analyzes the image acquired by the acquisition unit. The proposal unit proposes a product based on a diagnosis result obtained by the analysis unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

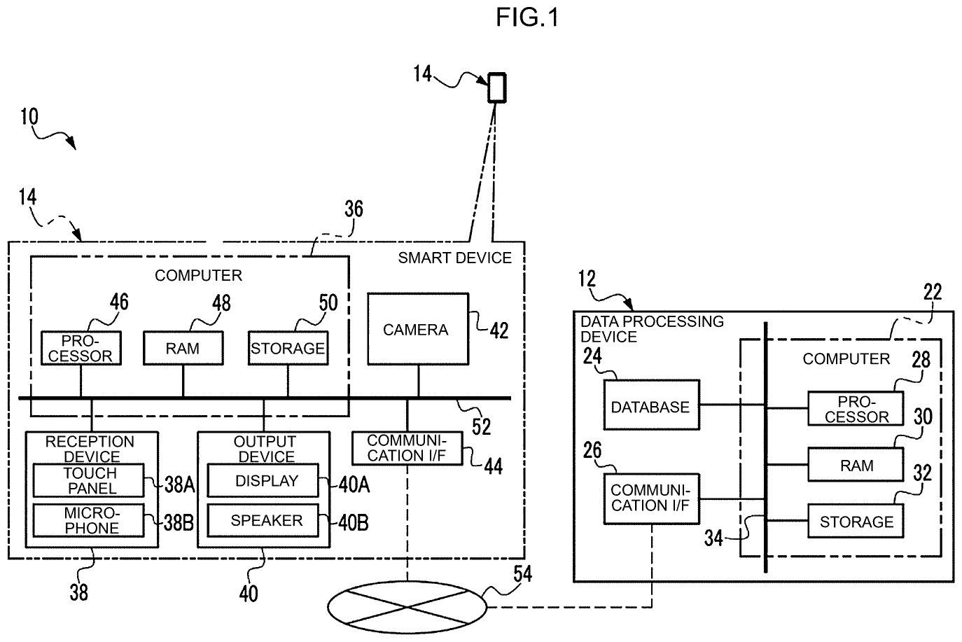

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

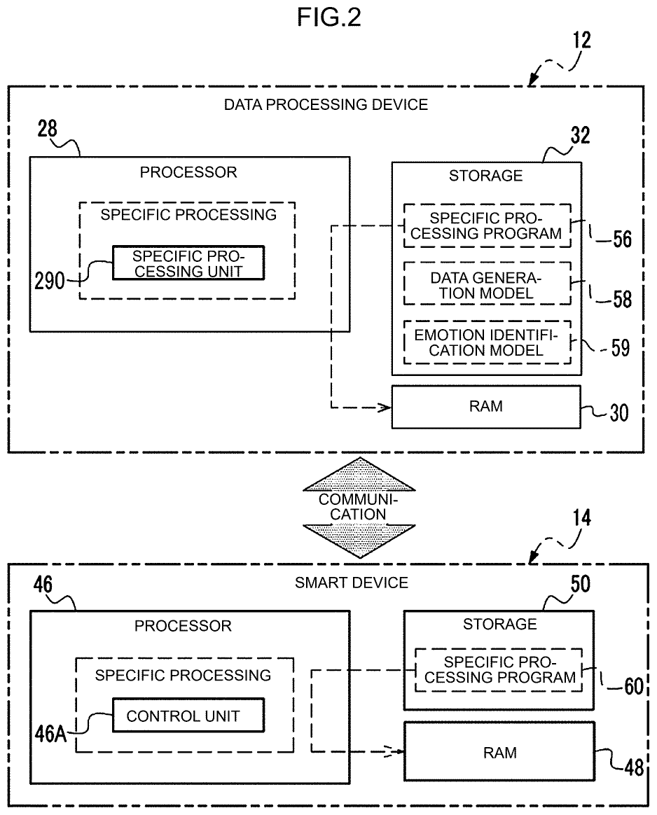

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The system according to the embodiment of the present invention is a system that performs image analysis of an emergency patient via AR glasses and introduces recommended products at a drugstore based on a simple diagnosis. This system acquires an image of the emergency patient, analyzes it using generative AI, and proposes products based on the diagnosis result. Furthermore, if first aid is required, the system provides the procedures for first aid. For example, the user wears AR glasses and acquires an image of the emergency patient. The AR glasses are equipped with a built-in camera, enabling real-time acquisition of images of the emergency patient. For instance, if the emergency patient has collapsed, the system can capture details such as posture and facial expression. Next, the acquired image is analyzed by generative AI. The generative AI analyzes the image of the emergency patient and diagnoses their condition. For example, the system can determine the condition of the emergency patient based on facial color, expression, and posture, thereby enabling rapid understanding of the emergency patient's condition. Based on the diagnosis result, recommended products available at a drugstore are presented. For example, if the emergency patient is suffering from dehydration, beverages for hydration are proposed. If symptoms such as headache or fever are present, antipyretics or analgesics are proposed. This allows the user to promptly provide appropriate products to the emergency patient. Furthermore, if first aid is required, the procedures are displayed to the user. For example, procedures for cardiopulmonary resuscitation or hemostasis are displayed on the AR glasses' display. This enables the user to promptly perform appropriate first aid. As a result, the system enables rapid understanding of the emergency patient's condition and provision of appropriate products. Additionally, since the system provides the procedures for first aid when necessary, the user can respond appropriately, thereby improving the survival rate of emergency patients and enabling prompt response. Specifically, the system uses a high-resolution camera module built into the AR glasses to acquire full-body images, close-up facial images, and continuous images from multiple angles (video stream, e.g., 1920×1080 pixels, 30 fps) in real time. The system first performs preprocessing on these image data, such as noise removal, contrast adjustment, and segmentation of facial and limb regions (using object detection models such as U-Net or YOLOv8), and then the image feature extraction unit generates multidimensional feature vectors (e.g., 512 dimensions) for facial color (RGB histogram, blood color estimation), expression (facial muscle patterns, action units), and posture (landmark extraction by skeleton estimation algorithms). These feature vectors are input into a multimodal neural network (e.g., a model combining ResNet-50 for image input and a Transformer-based encoder for text input), which outputs the condition of the emergency patient (e.g., dehydration, fever, consciousness disorder, trauma) as a probability distribution (e.g., a score of 0.0 to 1.0 for each symptom). For example, as input, an image tensor such as “pale facial color, agonized expression, supine posture” and speech recognition text such as “collapsed,” “unconscious” can be input simultaneously. As output, scores such as “dehydration: 0.85,” “fever: 0.10,” “trauma: 0.05” are obtained. The system uses a threshold judgment unit to determine these scores, and performs rule-based processing such as “if dehydration score is 0.7 or higher, recommend hydration beverage,” “if fever score is 0.5 or higher, recommend antipyretic.” Furthermore, the system matches the diagnosis result with a drugstore product database (structured data such as product name, ingredients, indications, inventory information) in the product proposal unit to generate an optimal product candidate list (e.g., oral rehydration solution, antipyretic analgesics, cooling sheets). The product proposal unit inputs the diagnosis result and user attributes (age, gender, medical history, etc.) into generative AI (e.g., LLM-based recommendation model) to generate recommendation texts with explanations, such as “Recommended product: oral rehydration solution, reason: strong dehydration symptoms, rapid hydration required.” Example outputs include “Recommended product: oral rehydration solution, recommended reason: strong suspicion of dehydration based on facial color and posture” and “Recommended product: antipyretic, recommended reason: high fever score.” These recommendation results are overlay-displayed on the AR glasses' display, and the user can select products or display additional information via voice commands or touchpad operations. If first aid is determined to be necessary, the system extracts the relevant procedures from a first aid procedure database (e.g., procedures for cardiopulmonary resuscitation, hemostasis, recovery position, etc., stored step-by-step in image, video, text, and audio formats) and sequentially displays guides such as “1. Start chest compressions,” “2. After 30 compressions, perform artificial respiration twice” on the AR glasses' display. It is also possible to guide the procedures by voice using a speech synthesis module. This enables the user to confirm the procedures visually and aurally and perform appropriate first aid. As a technical effect, the system, unlike conventional responses relying on human visual judgment and experience, integrates and analyzes diverse data such as images, audio, and text in a high-dimensional space, and realizes objective and rapid diagnosis, product recommendation, and first aid guidance by AI, thereby exhibiting remarkable effects such as improved survival rate, reduced risk of misdiagnosis, optimized product selection, and reduced user burden. Specific application fields include self-medication support in drugstore stores, initial response at emergency sites, family support in home care settings, and emergency response at event venues and public transportation.

The emergency patient response system according to the embodiment comprises an acquisition unit, an analysis unit, and a proposal unit. The acquisition unit acquires an image of the emergency patient. The image of the emergency patient may include, for example, still images, videos, X-ray images, and the like, but is not limited thereto. The acquisition unit may acquire an image of the emergency patient in real time using a camera built into AR glasses, for example. The acquisition unit may also acquire an image of the emergency patient using a camera of a smartphone or tablet. For example, the acquisition unit may capture details such as the posture and facial expression of the emergency patient when the patient has collapsed. The analysis unit analyzes the image acquired by the acquisition unit using generative AI. The analysis may be performed based on image processing algorithms or diagnostic criteria, but is not limited thereto. For example, the generative AI analyzes the facial color, expression, and posture of the emergency patient and diagnoses the condition. The generative AI may use a text-generating AI (e.g., LLM) to diagnose the condition of the emergency patient. The generative AI may also use a multimodal generative AI to analyze the image of the emergency patient. For example, the generative AI analyzes changes in facial color and distortion of expression to determine the condition of the emergency patient. The proposal unit proposes products based on the diagnosis result obtained by the analysis unit. The proposed products may include, for example, beverages for hydration, antipyretics, analgesics, and the like, but are not limited thereto. The proposal unit proposes products based on the diagnosis result using generative AI. For example, the generative AI proposes beverages for hydration if the emergency patient is suffering from dehydration. The generative AI proposes antipyretics or analgesics if the emergency patient exhibits symptoms such as headache or fever. Thus, the emergency patient response system according to the embodiment can acquire and analyze images of the emergency patient and propose products based on the diagnosis result. Some or all of the above-described processing in the proposal unit may be performed using AI or may be performed without using AI. For example, the proposal unit may use an AI model that receives the diagnosis result as input and outputs products to propose products. This enables product proposals according to the condition of the emergency patient. Furthermore, the proposal unit has a function to notify the user of the proposed products. For example, the proposal unit displays product information on the display of AR glasses. The proposal unit may also display product information on the screen of a smartphone or tablet. This enables the user to promptly provide appropriate products to the emergency patient. Specifically, the emergency patient response system uses AR glasses, smartphones, or tablet devices equipped with a high-resolution camera module (e.g., 1920×1080 pixels, 30 fps) as the acquisition unit to acquire full-body images, close-up facial images, and continuous images from multiple angles (video stream) in real time. The acquisition unit transmits the image data to a preprocessing unit, which performs noise removal (e.g., Gaussian filter), contrast adjustment, and segmentation of facial and limb regions (e.g., using object detection models such as YOLOv8 or U-Net). The preprocessed images are numerically converted into multidimensional feature vectors (e.g., 512 dimensions) for facial color (RGB histogram, blood color estimation), expression (facial muscle patterns, action units), and posture (landmark extraction by skeleton estimation algorithms) by a feature extraction unit. The analysis unit inputs these feature vectors into a multimodal neural network (e.g., a model combining ResNet-50 for image input and a Transformer-based encoder for text input) and outputs the condition of the emergency patient (e.g., dehydration, fever, consciousness disorder, trauma) as a probability distribution (e.g., a score of 0.0 to 1.0 for each symptom). As input to the AI, an image tensor such as “pale facial color, agonized expression, supine posture” (shape: 1×3×224×224) and speech recognition text such as “collapsed,” “unconscious” can be input simultaneously. As output from the AI, scores such as “dehydration: 0.85,” “fever: 0.10,” “trauma: 0.05” are obtained. The analysis unit uses a threshold judgment unit to determine these scores and performs rule-based processing such as “if dehydration score is 0.7 or higher, recommend hydration beverage,” “if fever score is 0.5 or higher, recommend antipyretic.” The proposal unit matches the diagnosis result with a drugstore product database (structured data such as product name, ingredients, indications, inventory information) to generate an optimal product candidate list (e.g., oral rehydration solution, antipyretic analgesics, cooling sheets). The product proposal unit inputs the diagnosis result and user attributes (age, gender, medical history, etc.) into generative AI (e.g., LLM-based recommendation model) to generate recommendation texts with explanations, such as “Recommended product: oral rehydration solution, reason: strong dehydration symptoms, rapid hydration required.” Example outputs include “Recommended product: oral rehydration solution, recommended reason: strong suspicion of dehydration based on facial color and posture” and “Recommended product: antipyretic, recommended reason: high fever score.” These recommendation results are overlay-displayed on the AR glasses' display, and the user can select products or display additional information via voice commands or touchpad operations. As a technical effect, the system, unlike conventional responses relying on human visual judgment and experience, integrates and analyzes diverse data such as images, audio, and text in a high-dimensional space, and realizes objective and rapid diagnosis and product recommendation by AI, thereby exhibiting remarkable effects such as improved survival rate, reduced risk of misdiagnosis, optimized product selection, and reduced user burden. Specific application fields include self-medication support in drugstore stores, initial response at emergency sites, family support in home care settings, and emergency response at event venues and public transportation.

The proposal unit can propose products based on the diagnosis result by means of generative AI. The proposal unit uses generative AI to propose products based on the diagnosis result. The generative AI may be, for example, a text-generating AI (such as LLM) or a multimodal generative AI, but is not limited thereto. The generative AI proposes products according to the condition of the emergency patient. For example, if the emergency patient is suffering from dehydration, the generative AI proposes beverages for hydration. If the emergency patient exhibits symptoms such as headache or fever, the generative AI proposes antipyretics or analgesics. The generative AI may use an AI model that receives the diagnosis result as input and outputs products to propose products. For example, the generative AI selects the optimal product based on the diagnosis result and notifies the user. Thus, by using generative AI, product proposals based on the diagnosis result are possible. Furthermore, the proposal unit has a function to notify the user of the proposed products. For example, the proposal unit displays product information on the display of AR glasses. The proposal unit may also display product information on the screen of a smartphone or tablet. This enables the user to promptly provide appropriate products to the emergency patient. Specifically, the proposal unit inputs symptom scores obtained as diagnosis results (e.g., “dehydration: 0.85,” “fever: 0.10,” etc.) and attribute information of the emergency patient (such as age, gender, medical history) into generative AI. The generative AI uses, for example, a Transformer-based large language model or a multimodal model that handles images and text simultaneously (e.g., image feature vectors plus text embeddings) to integratively analyze the input data in a high-dimensional space. The AI model matches the input with a product database (structured data such as product name, ingredients, indications, inventory information) to generate an optimal product candidate list. The AI output format is structured text such as recommended product name, recommended reason, and recommendation score (e.g., “Recommended product: oral rehydration solution, recommended reason: strong suspicion of dehydration, recommendation score: 0.92”). Example outputs include “Recommended product: oral rehydration solution, recommended reason: strong suspicion of dehydration based on facial color and posture” and “Recommended product: antipyretic, recommended reason: high fever score.” In subsequent processing, the proposal unit sends these recommendation results to the user interface unit for overlay display on the AR glasses' display or the screen of a smartphone or tablet. The user can select products or display additional information via voice commands or touchpad operations. As a technical effect, the proposal unit, unlike conventional responses relying on human experience and subjective judgment, realizes objective and rapid product recommendation by AI, thereby exhibiting remarkable effects such as optimized product selection, reduced risk of incorrect selection, and reduced user burden. Specific application fields include self-medication support in drugstore stores, initial response at emergency sites, family support in home care settings, and emergency response at event venues and public transportation.

The proposal unit can propose products corresponding to the condition of the emergency patient by means of generative AI. The proposal unit uses generative AI to propose products corresponding to the condition of the emergency patient. The generative AI may be, for example, a text-generating AI (such as LLM) or a multimodal generative AI, but is not limited thereto. The generative AI proposes products according to the condition of the emergency patient. For example, if the emergency patient is suffering from dehydration, the generative AI proposes beverages for hydration. If the emergency patient exhibits symptoms such as headache or fever, the generative AI proposes antipyretics or analgesics. The generative AI may use an AI model that receives the condition of the emergency patient as input and outputs products to propose products. For example, the generative AI selects the optimal product based on the condition of the emergency patient and notifies the user. Thus, product proposals corresponding to the condition of the emergency patient are possible. Furthermore, the proposal unit has a function to notify the user of the proposed products. For example, the proposal unit displays product information on the display of AR glasses. The proposal unit may also display product information on the screen of a smartphone or tablet. This enables the user to promptly provide appropriate products to the emergency patient. Specifically, the proposal unit inputs multidimensional feature vectors representing the condition of the emergency patient (e.g., numerical data for facial color, expression, posture, vital signs) and diagnosis results (e.g., “dehydration: 0.85,” “fever: 0.10,” etc.) into the AI model. The AI model uses, for example, a Transformer-based large language model or a multimodal model that handles images and text simultaneously to analyze the input data in a high-dimensional space and matches it with a product database (product name, ingredients, indications, inventory information, etc.). The AI output format is structured text such as recommended product name, recommended reason, and recommendation score (e.g., “Recommended product: oral rehydration solution, recommended reason: strong suspicion of dehydration, recommendation score: 0.92”). Example outputs include “Recommended product: oral rehydration solution, recommended reason: strong suspicion of dehydration based on facial color and posture” and “Recommended product: antipyretic, recommended reason: high fever score.” In subsequent processing, the proposal unit sends these recommendation results to the user interface unit for overlay display on the AR glasses' display or the screen of a smartphone or tablet. The user can select products or display additional information via voice commands or touchpad operations. As a technical effect, the proposal unit, unlike conventional responses relying on human experience and subjective judgment, realizes objective and rapid product recommendation by AI, thereby exhibiting remarkable effects such as optimized product selection, reduced risk of incorrect selection, and reduced user burden. Specific application fields include self-medication support in drugstore stores, initial response at emergency sites, family support in home care settings, and emergency response at event venues and public transportation.

The emergency patient response system comprises a display unit configured to display procedures for first aid. The display unit displays procedures for first aid. The procedures for first aid may include, for example, cardiopulmonary resuscitation, hemostasis, and the like, but are not limited thereto. The display unit may display procedures for first aid on the display of AR glasses, for example. The display unit may also display procedures for first aid on the screen of a smartphone or tablet. For example, the display unit may display the procedures for cardiopulmonary resuscitation step by step. The display unit may also display the procedures for hemostasis with illustrations. This enables the user to promptly perform appropriate first aid. Furthermore, the display unit has a function to guide the procedures for first aid by voice. For example, the display unit guides the procedures for cardiopulmonary resuscitation by voice. The display unit may also guide the procedures for hemostasis by voice. This enables the user to confirm the procedures for first aid both visually and aurally. By displaying the procedures for first aid, the user can perform appropriate first aid. Specifically, the display unit extracts the relevant procedures from a first aid procedure database (e.g., procedures for cardiopulmonary resuscitation, hemostasis, recovery position, etc., stored step by step in image, video, text, and audio formats) and sequentially displays guides such as “1. Start chest compressions,” “2. After 30 compressions, perform artificial respiration twice” on the display of AR glasses, smartphone, or tablet. It is also possible to guide the procedures by voice using a speech synthesis module. As input to the AI, structured data such as “Type of first aid: cardiopulmonary resuscitation,” “User attribute: beginner,” “Emergency patient condition: unconscious” can be provided. As output from the AI, a list of procedures such as “Step 1: Perform 30 chest compressions,” “Step 2: Perform artificial respiration twice,” or recommended procedures such as “Hemostasis: prioritize compression hemostasis” can be obtained. In subsequent processing, the display unit presents these procedures to the user in multiple modalities such as screen display, voice guidance, and illustrated display. As a technical effect, the display unit, unlike conventional paper manuals or oral explanations by humans, realizes situation-adaptive and individually optimized first aid guidance by AI, thereby exhibiting remarkable effects such as improved survival rate, reduced risk of incorrect operation, and reduced user burden. Specific application fields include initial response at emergency sites, family support in home care settings, emergency response at event venues and public transportation, and educational support for medical professionals.

The display unit can display procedures for first aid for cardiopulmonary resuscitation or hemostasis. The display unit displays procedures for first aid for cardiopulmonary resuscitation or hemostasis. The procedures for cardiopulmonary resuscitation may include, for example, the number and strength of chest compressions, methods of artificial respiration, and the like, but are not limited thereto. The procedures for hemostasis may include, for example, compression hemostasis, methods of using a tourniquet, and the like, but are not limited thereto. The display unit may display the procedures for cardiopulmonary resuscitation step by step on the display of AR glasses, for example. The display unit may also display the procedures for hemostasis with illustrations on the screen of a smartphone or tablet. This enables the user to promptly perform appropriate first aid. Furthermore, the display unit has a function to guide the procedures for first aid by voice. For example, the display unit guides the procedures for cardiopulmonary resuscitation by voice. The display unit may also guide the procedures for hemostasis by voice. This enables the user to confirm the procedures for first aid both visually and aurally. By displaying procedures for first aid such as cardiopulmonary resuscitation or hemostasis, the user can perform appropriate first aid. Specifically, the display unit extracts detailed procedures for “cardiopulmonary resuscitation” or “hemostasis” (e.g., 30 chest compressions, 2 artificial respirations, procedures for compression hemostasis, methods of applying a tourniquet, etc.) from the first aid procedure database and displays them step by step on the screen of AR glasses, smartphone, or tablet. It is also possible to guide the procedures by voice using a speech synthesis module. As input to the AI, structured data such as “Type of first aid: cardiopulmonary resuscitation,” “User attribute: beginner,” “Emergency patient condition: unconscious” can be provided. As output from the AI, a list of procedures such as “Step 1: Perform 30 chest compressions,” “Step 2: Perform artificial respiration twice,” or recommended procedures such as “Hemostasis: prioritize compression hemostasis” can be obtained. In subsequent processing, the display unit presents these procedures to the user in multiple modalities such as screen display, voice guidance, and illustrated display. As a technical effect, the display unit, unlike conventional paper manuals or oral explanations by humans, realizes situation-adaptive and individually optimized first aid guidance by AI, thereby exhibiting remarkable effects such as improved survival rate, reduced risk of incorrect operation, and reduced user burden. Specific application fields include initial response at emergency sites, family support in home care settings, emergency response at event venues and public transportation, and educational support for medical professionals.

The acquisition unit can estimate a user's emotion and adjust the timing of acquiring the image of the emergency patient based on the estimated emotion of the user. The acquisition unit estimates a user's emotion and adjusts the timing of acquiring the image of the emergency patient based on the estimated emotion. The estimation of the user's emotion may be realized using an emotion engine or generative AI, for example, by means of an emotion estimation function. The generative AI may be a text-generating AI (such as LLM) or a multimodal generative AI, but is not limited thereto. For example, if the user is nervous, the acquisition unit delays the timing of image acquisition and waits for the user to calm down. If the user is anxious, the acquisition unit may promptly acquire the image and start analysis. Furthermore, if the user is calm, the acquisition unit may adjust to acquire the image at the optimal timing. By adjusting the timing of image acquisition according to the user's emotion, images can be acquired at appropriate timing. Some or all of the above-described processing in the acquisition unit may be performed using AI or may be performed without using AI. For example, the acquisition unit may input the user's emotion data into generative AI and have the generative AI adjust the timing of image acquisition. Specifically, the acquisition unit simultaneously acquires the user's facial expression image (e.g., 1×3×224×224 RGB tensor), audio data (e.g., 5 seconds of audio waveform, 16 kHz sampling), and biometric sensor data (e.g., time-series vectors of heart rate, skin conductance, etc.), and inputs these into a multimodal neural network for emotion estimation (e.g., a model combining CNN for image input, RNN for audio input, and MLP for sensor input). The model integrates feature vectors extracted from each modality and outputs emotion labels such as “nervous,” “anxious,” “calm” as a probability distribution (e.g., nervous 0.7, anxious 0.2, calm 0.1). For example, if data such as “image of a tense face,” “fast speech,” and “heart rate above 100 bpm” are input, a high nervousness score is output. Example outputs include “nervous: 0.85,” “anxious: 0.10,” “calm: 0.05.” The acquisition unit uses a threshold judgment unit to determine these emotion scores and performs rule-based processing such as “if nervousness score is 0.7 or higher, delay image acquisition by 5 seconds,” “if anxiety score is 0.5 or higher, acquire immediately,” “if calmness score is 0.7 or higher, acquire at standard timing.” Furthermore, the adjustment of image acquisition timing is sequentially re-evaluated in real time while monitoring changes in the user's state, thereby minimizing the user's psychological burden and ensuring optimal image data. For training the AI model, a large-scale dataset with emotion labels (e.g., pairs of facial expressions, audio, biometric signals, and emotion labels) is used, and weights are optimized using a cross-entropy loss function. As a technical effect, the acquisition unit, unlike conventional image acquisition by simple timers or user-initiated operations, quantitatively estimates the user's psychological state in a high-dimensional feature space and realizes dynamic timing control by AI, thereby exhibiting remarkable effects such as improved image acquisition accuracy, reduced user stress, and reduced risk of incorrect operation. Specific application fields include stress response at emergency sites, family support in home care settings, simulation training in medical education, and emergency response in public spaces.

The acquisition unit can analyze a user's past emergency patient response history and select an optimal image acquisition method. The acquisition unit analyzes a user's past emergency patient response history and selects an optimal image acquisition method. The user's past emergency patient response history may include, for example, records of past first aid, diagnosis history, and the like, but is not limited thereto. For example, the acquisition unit may select an image acquisition method similar to a method that the user has successfully used in the past for emergency patient response. The acquisition unit may also avoid image acquisition methods that the user has failed with in the past and select a different method. Furthermore, the acquisition unit may select the most effective image acquisition method based on the user's past response history. By analyzing past response history, the optimal image acquisition method can be selected. Some or all of the above-described processing in the acquisition unit may be performed using AI or may be performed without using AI. For example, the acquisition unit may input the user's past emergency patient response history data into generative AI and have the generative AI select the optimal image acquisition method. Specifically, the acquisition unit refers to a database of emergency patient response history accumulated for each user (e.g., structured data such as date and time of first aid, diagnosis results, camera settings at the time of image acquisition, environmental conditions, success/failure flags), and inputs these history data as time-series vectors (e.g., 128-dimensional feature vectors for each response case) into the AI model. As input data, history such as “May 1, 2023: outdoors, clear weather, camera setting ISO400, success,” “Jun. 10, 2023: indoors, dim lighting, camera setting ISO800, failure” can be provided. The acquisition unit inputs these history data into a time-series analysis neural network such as LSTM or Transformer-based models to learn patterns of past successes and failures. The AI model calculates similarity to past history for a new case (e.g., current environment, user attributes, emergency patient condition) and outputs optimal image acquisition parameters (e.g., camera angle, exposure, burst mode, etc.). Example outputs from the AI include “Recommended camera setting: ISO400, shutter speed 1/60, prioritize close-up of face,” “Recommended acquisition method: two consecutive shots of full-body and close-up of face.” In subsequent processing, the acquisition unit automatically sets parameters in the camera control module based on the AI model's output and displays guidance to the user such as “Please shoot from this angle.” As a technical effect, the acquisition unit, unlike conventional uniform image acquisition procedures or user-initiated operations, analyzes each user's individual past history in a high-dimensional feature space and realizes personalized image acquisition optimization by AI, thereby exhibiting remarkable effects such as improved image quality, improved diagnostic accuracy, reduced user burden, and reduced risk of incorrect operation. Specific application fields include individualized image acquisition support at emergency sites, utilization of family response history in home care settings, guidance for shooting based on training history in medical education, and feedback utilization of emergency response history in public spaces.

The acquisition unit can perform filtering based on the user's current location information or environment when acquiring an image of the emergency patient. The acquisition unit performs filtering based on the user's current location information or environment when acquiring an image of the emergency patient. Location information may include, for example, GPS data, location information services, and the like, but is not limited thereto. Environment may include, for example, ambient temperature, humidity, noise level, and the like, but is not limited thereto. For example, if the user is outdoors, the acquisition unit acquires images considering the ambient light. If the user is indoors, the acquisition unit may acquire images considering the lighting conditions. Furthermore, the acquisition unit may select the optimal image acquisition method based on the user's location information. By performing filtering based on location information or environment, appropriate images can be acquired. Some or all of the above-described processing in the acquisition unit may be performed using AI or may be performed without using AI. For example, the acquisition unit may input the user's location information or environment data into generative AI and have the generative AI perform filtering. Specifically, the acquisition unit collects location information such as latitude, longitude, altitude, and indoor/outdoor determination obtained from GPS modules, Wi-Fi positioning, Bluetooth beacons, etc. (e.g., latitude 35.6895, longitude 139.6917, outdoor flag 1), and multidimensional environment data such as temperature (e.g., 28.5° C.), humidity (e.g., 60%), illuminance (e.g., 500 lx), noise level (e.g., 65 dB) obtained from environment sensors in real time. The acquisition unit inputs these location and environment data as 128-dimensional feature vectors into the AI model and infers optimal filtering conditions for image acquisition (e.g., exposure adjustment, automatic white balance, noise reduction strength). As input to the AI, data such as “outdoors, clear weather, illuminance 2000 lx,” “indoors, fluorescent light, illuminance 300 lx, noise 80 dB” can be provided. Example outputs from the AI include “Exposure +1.0, ISO200, strong noise reduction,” “White balance: fluorescent light mode.” In subsequent processing, the acquisition unit automatically sets parameters in the camera control module based on the AI model's output and displays guidance to the user such as “Adjust the angle due to backlight.” As a technical effect, the acquisition unit, unlike conventional uniform camera settings or user-initiated operations, analyzes real-time location and environment data in a high-dimensional feature space and realizes dynamic image acquisition optimization by AI, thereby exhibiting remarkable effects such as improved image quality, improved diagnostic accuracy, reduced user burden, and reduced risk of incorrect shooting. Specific application fields include emergency response at outdoor event venues, adaptation to environmental changes in home care settings, optimization of image acquisition under various lighting conditions in medical settings, and response to noisy environments in public transportation.

The acquisition unit can estimate a user's emotion and determine a priority order of images to be acquired based on the estimated emotion of the user. The acquisition unit estimates a user's emotion and determines a priority order of images to be acquired based on the estimated emotion. The estimation of the user's emotion may be realized using an emotion engine or generative AI, for example, by means of an emotion estimation function. The generative AI may be a text-generating AI (such as LLM) or a multimodal generative AI, but is not limited thereto. For example, if the user is nervous, the acquisition unit prioritizes acquisition of images of important parts. If the user is relaxed, the acquisition unit may acquire images of the whole in a balanced manner. Furthermore, if the user is anxious, the acquisition unit may prioritize acquisition of images of the emergency patient's face and expression. By determining the priority order of images according to the user's emotion, important images can be acquired preferentially. Some or all of the above-described processing in the acquisition unit may be performed using AI or may be performed without using AI. For example, the acquisition unit may input the user's emotion data into generative AI and have the generative AI determine the priority order of images. Specifically, the acquisition unit simultaneously acquires the user's facial expression image (e.g., 1×3×224×224 RGB tensor), audio data (e.g., 5 seconds of audio waveform, 16 kHz sampling), and biometric sensor data (e.g., time-series vectors of heart rate, skin conductance, etc.), and inputs these into a multimodal neural network for emotion estimation (e.g., a model combining CNN for image input, RNN for audio input, and MLP for sensor input). The model integrates feature vectors extracted from each modality and outputs emotion labels such as “nervous,” “anxious,” “calm” as a probability distribution (e.g., nervous 0.7, anxious 0.2, calm 0.1). For example, if data such as “image of a tense face,” “fast speech,” and “heart rate above 100 bpm” are input, a high nervousness score is output. Example outputs include “nervous: 0.85,” “anxious: 0.10,” “calm: 0.05.” The acquisition unit uses a threshold judgment unit to determine these emotion scores and performs rule-based processing such as “if nervousness score is 0.7 or higher, prioritize acquisition of close-up images of face and chest,” “if calmness score is 0.7 or higher, acquire full-body and surrounding environment images in a balanced manner.” In subsequent processing, the image acquisition priority list (e.g., face>chest>full body>limbs) is sent to the camera control module, and guidance such as “Please shoot the face first” is displayed to the user. As a technical effect, the acquisition unit, unlike conventional uniform image acquisition order or user-initiated operations, quantitatively estimates the user's psychological state in a high-dimensional feature space and realizes dynamic image acquisition priority control by AI, thereby exhibiting remarkable effects such as prevention of missing important parts, improved diagnostic accuracy, reduced user stress, and reduced risk of incorrect operation. Specific application fields include stress response at emergency sites, family support in home care settings, simulation training in medical education, and emergency response in public spaces.

The acquisition unit can analyze audio information around the user when acquiring an image of the emergency patient and preferentially acquire related images. The acquisition unit analyzes audio information around the user when acquiring an image of the emergency patient and preferentially acquires related images. Audio information may include, for example, use of microphones, speech recognition technology, and the like, but is not limited thereto. For example, the acquisition unit detects the emergency patient's breathing sounds from surrounding audio and prioritizes acquisition of images of that part. The acquisition unit may also detect the emergency patient's voice from surrounding audio and prioritize acquisition of images of that part. Furthermore, the acquisition unit may analyze environmental sounds from surrounding audio and prioritize acquisition of images related to the emergency patient's condition. By analyzing surrounding audio information, related images can be acquired preferentially. Some or all of the above-described processing in the acquisition unit may be performed using AI or may be performed without using AI. For example, the acquisition unit may input surrounding audio data into generative AI and have the generative AI preferentially acquire related images. Specifically, the acquisition unit acquires audio data (e.g., 16 kHz sampling, 5 seconds of audio waveform) in real time from microphones built into AR glasses or smartphones, and uses a speech recognition and acoustic feature extraction module (e.g., MFCC extraction, spectrogram generation) to convert the data into feature vectors. The acquisition unit inputs these audio feature vectors into a neural network for audio event detection (e.g., CNN+RNN hybrid model) and outputs audio event labels such as “breathing sound,” “groaning,” “environmental noise” as a probability distribution (e.g., breathing sound 0.8, groaning 0.6, noise 0.2). As input to the AI, data such as “audio waveform with periodic detection of breathing sound,” “audio with mixed groaning” can be provided. Example outputs from the AI include “breathing sound: 0.85,” “groaning: 0.60,” “environmental noise: 0.10.” The acquisition unit uses a threshold judgment unit to determine these audio event scores and performs rule-based processing such as “if breathing sound score is 0.7 or higher, prioritize acquisition of chest images,” “if groaning score is 0.5 or higher, prioritize acquisition of face and mouth images.” In subsequent processing, the image acquisition priority list (e.g., chest>face>full body) is sent to the camera control module, and guidance such as “Please shoot the chest” is displayed to the user. As a technical effect, the acquisition unit, unlike conventional image acquisition using only visual information, analyzes audio information in a high-dimensional feature space and realizes multimodal image acquisition priority control by AI, thereby exhibiting remarkable effects such as prevention of missing important parts, improved diagnostic accuracy, reduced user burden, and reduced risk of incorrect operation. Specific application fields include support for detection of abnormal breathing at emergency sites, audio event-linked shooting in home care settings, simulation training with audio linkage in medical education, and emergency response in public spaces.

The acquisition unit can analyze a user's online activity when acquiring an image of the emergency patient and acquire related images. The acquisition unit analyzes a user's online activity when acquiring an image of the emergency patient and acquires related images. Online activity may include, for example, social media posts, browsing history, and the like, but is not limited thereto. For example, the acquisition unit may acquire information related to the emergency patient's condition from the user's social media posts and preferentially acquire related images. The acquisition unit may also acquire images of the emergency patient based on location information from the user's social media. Furthermore, the acquisition unit may analyze the user's social media friend relationships and acquire images related to the emergency patient. By analyzing social media activity, related images can be acquired. Some or all of the above-described processing in the acquisition unit may be performed using AI or may be performed without using AI. For example, the acquisition unit may input the user's online activity data into generative AI and have the generative AI acquire related images. Specifically, the acquisition unit collects the user's social media post data (e.g., post text, images, location information, post time, friend tags as structured data) and browsing history (e.g., page URLs viewed, search keywords, access times), and uses a natural language processing module (e.g., BERT-based text encoder) and a graph neural network (for friend relationship network analysis) to convert the data into feature vectors. The acquisition unit inputs these online activity feature vectors into the AI model and outputs the emergency patient's condition and relevance scores (e.g., degree of match between post content and emergency patient symptoms, degree of match of location information, degree of relevance of friend relationships). As input to the AI, data such as “Post text: ‘My friend collapsed’,” “Location: Shinjuku Station,” “Friend tag: Mr. A” can be provided. Example outputs from the AI include “Relevance score: 0.92 (high probability that Mr. A is the emergency patient),” “Location match: 0.85.” The acquisition unit uses a threshold judgment unit to determine these relevance scores and performs rule-based processing such as “if relevance score is 0.8 or higher, prioritize acquisition of face images of the relevant person,” “if location match is 0.7 or higher, acquire images of the surrounding area.” In subsequent processing, the image acquisition priority list is sent to the camera control module, and guidance such as “Please shoot the face of Mr. A” is displayed to the user. As a technical effect, the acquisition unit, unlike conventional image acquisition using only on-site information, analyzes online activity data in a high-dimensional feature space and realizes multi-source collaborative image acquisition optimization by AI, thereby exhibiting remarkable effects such as improved accuracy in identifying related persons and sites, improved diagnostic accuracy, reduced user burden, and reduced risk of incorrect shooting. Specific application fields include SNS-linked emergency response at event venues, utilization of family networks in home care settings, linkage with patient history in medical settings, and identification of emergency patients in crowds in public spaces.

The analysis unit can estimate a user's emotion and adjust the accuracy of image analysis based on the estimated emotion of the user. The analysis unit estimates a user's emotion and adjusts the accuracy of image analysis based on the estimated emotion. The estimation of the user's emotion may be realized using an emotion engine or generative AI, for example, by means of an emotion estimation function. The generative AI may be a text-generating AI (such as LLM) or a multimodal generative AI, but is not limited thereto. For example, if the user is nervous, the analysis unit increases the accuracy of image analysis and provides diagnosis results quickly. If the user is relaxed, the analysis unit may perform image analysis with normal accuracy. Furthermore, if the user is anxious, the analysis unit may increase the accuracy of image analysis and provide diagnosis results quickly. By adjusting the accuracy of image analysis according to the user's emotion, diagnosis results can be provided quickly. Some or all of the above-described processing in the analysis unit may be performed using AI or may be performed without using AI. For example, the analysis unit may input the user's emotion data into generative AI and have the generative AI adjust the accuracy of image analysis. Specifically, the analysis unit simultaneously acquires facial expression images (e.g., 1×3×224×224 RGB tensor), audio data (e.g., 5 seconds of audio waveform, 16 kHz sampling), and biometric sensor data (e.g., time-series vectors of heart rate, skin conductance, etc.) for emotion estimation, and inputs these into a multimodal neural network for emotion estimation (combining CNN for image input, RNN for audio input, and MLP for sensor input). The model integrates feature vectors extracted from each modality and outputs emotion labels such as “nervous,” “anxious,” “calm” as a probability distribution (e.g., nervous 0.7, anxious 0.2, calm 0.1). For example, if data such as “image of a tense face,” “fast speech,” and “heart rate above 100 bpm” are input, a high nervousness score is output. Example outputs include “nervous: 0.85,” “anxious: 0.10,” “calm: 0.05.” The analysis unit uses a threshold judgment unit to determine these emotion scores and performs rule-based processing such as “if nervousness score is 0.7 or higher, automatically set image analysis accuracy parameters (e.g., threshold for object detection model, number of ensemble inferences, strength of image preprocessing) to high-accuracy mode,” “if calmness score is 0.7 or higher, analyze in standard mode.” Image analysis itself is performed by combining feature extraction using CNNs such as ResNet-50 or EfficientNet, object detection using YOLOv8, segmentation using U-Net, and multimodal integration models based on Transformers. As input to the AI, an image tensor such as “pale facial color, agonized expression, supine posture” and speech recognition text such as “collapsed,” “unconscious” can be input simultaneously. As output from the AI, scores such as “dehydration: 0.85,” “fever: 0.10,” “trauma: 0.05” are obtained. For accuracy adjustment, parameters such as increasing the number of inferences for ensemble averaging, increasing the strength of noise removal in image preprocessing, and tightening thresholds are automatically controlled. In subsequent processing, the diagnosis result is sent to the product proposal unit or first aid guide unit, and optimal information is presented according to the user's situation. As a technical effect, the analysis unit, unlike conventional uniform image analysis accuracy settings or operations relying on human subjective judgment, quantitatively estimates the user's psychological state in a high-dimensional feature space and realizes dynamic image analysis accuracy control by AI, thereby exhibiting remarkable effects such as improved diagnostic accuracy, reduced risk of misdiagnosis, reduced user stress, and improved system responsiveness. Specific application fields include stress response at emergency sites, family support in home care settings, simulation training in medical education, and emergency response in public spaces.

The analysis unit can refer to past health data of the emergency patient during image analysis to improve the accuracy of the analysis. The analysis unit refers to past health data of the emergency patient during image analysis to improve the accuracy of the analysis. Past health data may include, for example, electronic medical records, health checkup results, and the like, but are not limited thereto. For example, the analysis unit may refer to past health checkup data of the emergency patient to improve the accuracy of image analysis. The analysis unit may also refer to the emergency patient's past medical history to improve the accuracy of image analysis. Furthermore, the analysis unit may refer to the emergency patient's past treatment history to improve the accuracy of image analysis. By referring to past health data, the accuracy of image analysis can be improved. Some or all of the above-described processing in the analysis unit may be performed using AI or may be performed without using AI. For example, the analysis unit may input the emergency patient's past health data into generative AI and have the generative AI improve the accuracy of analysis. Specifically, the analysis unit obtains electronic medical record data of the emergency patient (e.g., diagnosis name, past illnesses, medication history, allergy information, time-series data of test values), health checkup results (e.g., blood test values, ECG waveforms, imaging results), and treatment history (e.g., surgical history, hospitalization history, treatment responsiveness) from a structured database, and inputs these as feature vectors (e.g., 128-dimensional numerical vectors for each item) into the AI model. The analysis unit simultaneously inputs image data (e.g., 1×3×224×224 RGB tensor) and health data vectors into a multimodal neural network (e.g., ResNet-50 for image input, MLP for health data input, Transformer encoder for integration) and outputs the condition of the emergency patient (e.g., dehydration, fever, consciousness disorder, trauma) as a probability distribution (e.g., a score of 0.0 to 1.0 for each symptom). As input to the AI, data such as “Image: pale facial color, supine posture,” “Health data: diabetes in the past, recent HbA1c 8.2%,” “Treatment history: under treatment for hypertension” can be provided. Example outputs from the AI include “dehydration: 0.85,” “fever: 0.10,” “consciousness disorder: 0.30.” The analysis unit uses a threshold judgment unit to determine these scores and sends the diagnosis result to the product proposal unit or first aid guide unit. For training the AI model, pairs of image, health data, and diagnosis labels are used, and cross-entropy loss functions and multitask learning are applied. As a technical effect, the analysis unit, unlike conventional image-only analysis or diagnosis relying on human experience, integratively analyzes past health data in a high-dimensional feature space and realizes individually optimized diagnostic accuracy improvement by AI, thereby exhibiting remarkable effects such as reduced risk of misdiagnosis, improved diagnostic accuracy, reduced user burden, and improved medical safety. Specific application fields include diagnosis considering past medical history at emergency sites, utilization of family history in home care settings, diagnosis linked to electronic medical records in medical settings, and emergency response in public spaces.

The analysis unit can perform analysis based on current environment information of the emergency patient during image analysis. The analysis unit performs analysis based on current environment information of the emergency patient during image analysis. Current environment information may include, for example, ambient temperature, humidity, noise level, and the like, but is not limited thereto. For example, if the emergency patient is outdoors, the analysis unit performs image analysis considering the environment information. If the emergency patient is indoors, the analysis unit may perform image analysis considering the environment information. Furthermore, the analysis unit may perform image analysis considering environment information such as ambient temperature and humidity around the emergency patient. By considering current environment information, appropriate image analysis can be performed. Some or all of the above-described processing in the analysis unit may be performed using AI or may be performed without using AI. For example, the analysis unit may input the emergency patient's current environment information into generative AI and have the generative AI perform analysis. Specifically, the analysis unit inputs multidimensional environment data such as temperature (e.g., 28.5° C.), humidity (e.g., 60%), illuminance (e.g., 500 lx), noise level (e.g., 65 dB), and indoor/outdoor determination (e.g., by GPS, Wi-Fi, Bluetooth beacon) obtained from environment sensors as 128-dimensional feature vectors into the AI model. The analysis unit simultaneously inputs image data (e.g., 1×3×224×224 RGB tensor) and environment data vectors into a multimodal neural network (e.g., ResNet-50 for image input, MLP for environment data input, Transformer encoder for integration) and outputs the condition of the emergency patient (e.g., heatstroke, hypothermia, trauma) as a probability distribution (e.g., a score of 0.0 to 1.0 for each symptom). As input to the AI, data such as “Image: red facial color, sweating,” “Environment: outdoors, temperature 35° C., humidity 80%,” “Image: pale facial color, shivering,” “Environment: outdoors, temperature 5° C., humidity 30%” can be provided. Example outputs from the AI include “heatstroke: 0.90,” “dehydration: 0.80,” “hypothermia: 0.85.” The analysis unit uses a threshold judgment unit to determine these scores and sends the diagnosis result to the product proposal unit or first aid guide unit. For training the AI model, pairs of image, environment data, and diagnosis labels are used, and cross-entropy loss functions and multitask learning are applied. As a technical effect, the analysis unit, unlike conventional image-only analysis or diagnosis relying on human experience, integratively analyzes real-time environment data in a high-dimensional feature space and realizes situation-adaptive diagnostic accuracy improvement by AI, thereby exhibiting remarkable effects such as reduced risk of misdiagnosis, improved diagnostic accuracy, reduced user burden, and improved adaptability to the site. Specific application fields include diagnosis of heatstroke and hypothermia at outdoor event venues, adaptation to environmental changes in home care settings, optimization of diagnosis under various lighting and noise conditions in medical settings, and emergency response in public transportation.

The analysis unit can estimate a user's emotion and adjust a display method of analysis results based on the estimated emotion of the user. The analysis unit estimates a user's emotion and adjusts a display method of analysis results based on the estimated emotion. The estimation of the user's emotion may be realized using an emotion engine or generative AI, for example, by means of an emotion estimation function. The generative AI may be a text-generating AI (such as LLM) or a multimodal generative AI, but is not limited thereto. For example, if the user is nervous, the analysis unit provides a simple and highly visible display method. If the user is relaxed, the analysis unit may provide a display method including detailed information. Furthermore, if the user is in a hurry, the analysis unit may provide a display method focusing on key points. By adjusting the display method according to the user's emotion, highly visible display is possible. Some or all of the above-described processing in the analysis unit may be performed using AI or may be performed without using AI. For example, the analysis unit may input the user's emotion data into generative AI and have the generative AI adjust the display method. Specifically, the analysis unit inputs the user's facial expression image (e.g., 1×3×224×224 RGB tensor), audio data (e.g., 5 seconds of audio waveform, 16 kHz sampling), and biometric sensor data (e.g., time-series vectors of heart rate, skin conductance, etc.) into a multimodal neural network for emotion estimation and outputs emotion labels such as “nervous,” “anxious,” “calm” as a probability distribution. As input to the AI, data such as “image of a tense face,” “fast speech,” and “heart rate above 100 bpm” can be provided, resulting in a high nervousness score. Example outputs from the AI include “nervous: 0.85,” “anxious: 0.10,” “calm: 0.05.” The analysis unit uses a threshold judgment unit to determine these emotion scores and performs rule-based processing such as “if nervousness score is 0.7 or higher, display analysis results in large font, simple colors, and short sentences with only key points,” “if calmness score is 0.7 or higher, display detailed numerical values, graphs, and supplementary explanations.” The adjustment of the display method is transmitted to the user interface unit as display layout parameters (e.g., font size, color, amount of information, display order), and the analysis results are presented in an optimized form on the screen of AR glasses, smartphone, or tablet. As a technical effect, the analysis unit, unlike conventional uniform information display or operations relying on human subjective judgment, quantitatively estimates the user's psychological state in a high-dimensional feature space and realizes dynamic optimization of the display method by AI, thereby exhibiting remarkable effects such as improved visibility, improved information transmission efficiency, reduced user stress, and reduced risk of misunderstanding. Specific application fields include stress response at emergency sites, family support in home care settings, simulation training in medical education, and emergency response in public spaces.

The analysis unit can perform analysis based on a geographical distribution of emergency patients during image analysis. The analysis unit performs analysis based on a geographical distribution of emergency patients during image analysis. Geographical distribution may include, for example, the number of patients in each region, frequency of occurrence, and the like, but is not limited thereto. For example, if emergency patients are concentrated in a specific region, the analysis unit performs image analysis considering the characteristics of that region. If emergency patients are distributed over a wide area, the analysis unit may perform image analysis considering the geographical distribution. Furthermore, the analysis unit may select the optimal analysis method based on the geographical distribution of emergency patients. By considering geographical distribution, appropriate image analysis can be performed. Some or all of the above-described processing in the analysis unit may be performed using AI or may be performed without using AI. For example, the analysis unit may input geographical distribution data of emergency patients into generative AI and have the generative AI perform analysis. Specifically, the analysis unit obtains location information of emergency patients (e.g., GPS latitude and longitude, prefecture/municipality codes), data on frequency of patient occurrence in each region (e.g., number of cases in the past week, trend of outbreaks), and regional characteristic data (e.g., temperature, humidity, population density, number of medical institutions) from a structured database, and inputs these as feature vectors (e.g., 128 dimensions) into the AI model. The analysis unit simultaneously inputs image data (e.g., 1×3×224×224 RGB tensor) and geographical distribution vectors into a multimodal neural network (e.g., ResNet-50 for image input, MLP for geographical data input, Transformer encoder for integration) and outputs the condition of the emergency patient (e.g., infectious disease, heatstroke, trauma) as a probability distribution (e.g., a score of 0.0 to 1.0 for each symptom). As input to the AI, data such as “Image: rash present,” “Location: Shinjuku-ku, Tokyo,” “Regional frequency: measles outbreak” can be provided. Example outputs from the AI include “infectious disease: 0.90,” “heatstroke: 0.10.” The analysis unit uses a threshold judgment unit to determine these scores and sends the diagnosis result to the product proposal unit or first aid guide unit. For training the AI model, pairs of image, geographical data, and diagnosis labels are used, and cross-entropy loss functions and multitask learning are applied. As a technical effect, the analysis unit, unlike conventional image-only analysis or diagnosis relying on human experience, integratively analyzes geographical distribution data in a high-dimensional feature space and realizes region-adaptive diagnostic accuracy improvement by AI, thereby exhibiting remarkable effects such as reduced risk of misdiagnosis, improved diagnostic accuracy, early detection of epidemic diseases, and reduced user burden. Specific application fields include diagnostic support in epidemic regions, analysis of patient distribution at disaster sites, diagnosis considering regional characteristics in home care settings, and emergency response in public spaces.

The analysis unit can refer to related literature of the emergency patient during image analysis to improve the accuracy of the analysis. The analysis unit refers to related literature of the emergency patient during image analysis to improve the accuracy of the analysis. Related literature may include, for example, medical papers, guidelines, and the like, but is not limited thereto. For example, the analysis unit may refer to the latest research papers related to the condition of the emergency patient to improve the accuracy of image analysis. The analysis unit may also refer to past literature related to the symptoms of the emergency patient to improve the accuracy of image analysis. Furthermore, the analysis unit may refer to literature related to the treatment of the emergency patient to improve the accuracy of image analysis. By referring to related literature, the accuracy of image analysis can be improved. Some or all of the above-described processing in the analysis unit may be performed using AI or may be performed without using AI. For example, the analysis unit may input related literature data of the emergency patient into generative AI and have the generative AI improve the accuracy of analysis. Specifically, the analysis unit obtains text data (e.g., paper titles, abstracts, main text, figure captions) from medical literature databases (e.g., PubMed, guideline collections, case report collections) and uses a natural language processing module (e.g., BERT-based text encoder) to convert the data into feature vectors, which are input together with image data (e.g., 1×3×224×224 RGB tensor) into a multimodal neural network (e.g., ResNet-50 for image input, Transformer encoder for text input, cross-attention layer for integration). The analysis unit outputs the condition of the emergency patient (e.g., dehydration, fever, rash, trauma) as a probability distribution (e.g., a score of 0.0 to 1.0 for each symptom). As input to the AI, data such as “Image: rash present,” “Abstract: diagnostic criteria for infectious diseases with rash and fever” can be provided. Example outputs from the AI include “infectious disease: 0.92,” “allergic reaction: 0.10.” The analysis unit uses a threshold judgment unit to determine these scores and sends the diagnosis result to the product proposal unit or first aid guide unit. For training the AI model, pairs of image, literature data, and diagnosis labels are used, and cross-entropy loss functions and multitask learning are applied. As a technical effect, the analysis unit, unlike conventional image-only analysis or diagnosis relying on human experience, integratively analyzes the latest medical knowledge in a high-dimensional feature space and realizes knowledge-extended diagnostic accuracy improvement by AI, thereby exhibiting remarkable effects such as reduced risk of misdiagnosis, improved diagnostic accuracy, reflection of the latest treatment methods, and reduced user burden. Specific application fields include response to emerging diseases at emergency sites, reflection of the latest guidelines in home care settings, case-based diagnostic support in medical settings, and emergency response in public spaces.

The proposal unit can estimate a user's emotion and adjust a method of expressing product proposals based on the estimated emotion of the user. The proposal unit estimates a user's emotion and adjusts a method of expressing product proposals based on the estimated emotion. The estimation of the user's emotion may be realized using an emotion engine or generative AI, for example, by means of an emotion estimation function. The generative AI may be a text-generating AI (such as LLM) or a multimodal generative AI, but is not limited thereto. For example, if the user is nervous, the proposal unit provides simple and highly visible product proposals. If the user is relaxed, the proposal unit may provide product proposals including detailed information. Furthermore, if the user is in a hurry, the proposal unit may provide product proposals focusing on key points. By adjusting the method of expression according to the user's emotion, highly visible product proposals are possible. Some or all of the above-described processing in the proposal unit may be performed using AI or may be performed without using AI. For example, the proposal unit may input the user's emotion data into generative AI and have the generative AI adjust the method of expression. Specifically, the proposal unit simultaneously acquires facial expression images (e.g., 1×3×224×224 RGB tensor), audio data (e.g., 5 seconds of audio waveform, 16 kHz sampling), and biometric sensor data (e.g., time-series vectors of heart rate, skin conductance, etc.) for emotion estimation, and inputs these into a multimodal neural network for emotion estimation (combining CNN for image input, RNN for audio input, and MLP for sensor input). The model integrates feature vectors extracted from each modality and outputs emotion labels such as “nervous,” “relaxed,” “in a hurry” as a probability distribution (e.g., nervous 0.7, relaxed 0.2, in a hurry 0.1). For example, if data such as “image of a tense face,” “fast speech,” and “heart rate above 100 bpm” are input, a high nervousness score is output. Example outputs from the AI include “nervous: 0.85,” “relaxed: 0.10,” “in a hurry: 0.05.” The proposal unit uses a threshold judgment unit to determine these emotion scores and performs rule-based processing such as “if nervousness score is 0.7 or higher, display product proposal text in large font, simple colors, and short sentences with only key points,” “if relaxed score is 0.7 or higher, display detailed product descriptions, ingredient information, and usage precautions,” “if in a hurry score is 0.7 or higher, emphasize only the most important product name and reason.” The product proposal itself is output as structured text such as recommended product name, recommended reason, and recommendation score (e.g., “Recommended product: oral rehydration solution, recommended reason: strong suspicion of dehydration, recommendation score: 0.92”) by matching diagnosis results and user attributes with a product database (product name, ingredients, indications, inventory information, etc.). In subsequent processing, the proposal unit sends these recommendation results and expression method parameters to the user interface unit, and product proposals are presented in an optimized form on the screen of AR glasses, smartphone, or tablet. The user can select products or display additional information via voice commands or touchpad operations. As a technical effect, the proposal unit, unlike conventional uniform product proposal display or operations relying on human subjective judgment, quantitatively estimates the user's psychological state in a high-dimensional feature space and realizes dynamic optimization of product proposal expression by AI, thereby exhibiting remarkable effects such as improved visibility, improved information transmission efficiency, reduced user stress, and reduced risk of misunderstanding. Specific application fields include stress response at emergency sites, self-medication support in drugstore stores, family support in home care settings, and emergency response at event venues and public transportation.

The proposal unit can adjust the level of detail of proposals based on the condition of the emergency patient when proposing a product. The proposal unit adjusts the level of detail of proposals based on the condition of the emergency patient when proposing a product. The condition of the emergency patient may include, for example, vital signs and severity of symptoms, but is not limited thereto. For example, when the condition of the emergency patient is severe, the proposal unit provides a detailed product proposal. When the condition of the emergency patient is mild, the proposal unit may provide a concise product proposal. Furthermore, the proposal unit may provide an optimal product proposal according to the condition of the emergency patient. By adjusting the level of detail of proposals according to the condition of the emergency patient, appropriate product proposals can be made. Some or all of the above-described processing in the proposal unit may be performed using AI or without using AI. For example, the proposal unit may input condition data of the emergency patient into generative AI and have the generative AI adjust the level of detail of the proposal. Specifically, the proposal unit inputs vital signs of the emergency patient (e.g., numerical data such as heart rate, blood pressure, body temperature, respiratory rate), symptom scores (e.g., probability values such as “dehydration: 0.85”, “fever: 0.10”), and consciousness level (e.g., scores such as JCS, GCS) as input data to the AI model. The AI model encodes these condition data as a multidimensional feature vector (e.g., 128 dimensions) and outputs labels such as “severe”, “moderate”, “mild” as a probability distribution (e.g., severe 0.8, moderate 0.1, mild 0.1) in the severity determination unit. Examples of input to the AI include “heart rate 120 bpm, blood pressure 80/50 mmHg, consciousness level JCS3” and “body temperature 37.2° C., mild headache”. Examples of AI output include “severity: 0.90 (severe)” and “severity: 0.20 (mild)”. The proposal unit determines the severity score in the threshold determination unit and performs rule-based processing such as “if the severity score is 0.7 or higher, add detailed explanations, usage precautions, side effect information, emergency contact information, etc. to the product proposal text” and “if the mild score is 0.7 or higher, display only the product name and a brief recommendation reason”. The product proposal itself uses the diagnosis result and user attributes as input, matches them with the product database (product name, ingredients, indications, inventory information, etc.), and outputs structured text such as recommended product name, recommendation reason, and recommendation score (e.g., “Recommended product: oral rehydration solution, recommendation reason: dehydration is strongly suspected, recommendation score: 0.92”). In subsequent processing, the proposal unit sends these recommendation results and detail level parameters to the user interface unit and presents the product proposal in a form optimized for AR glasses, smartphones, or tablet screens. As a technical effect, the proposal unit, unlike conventional uniform product proposal displays or operations dependent on human subjective judgment, quantitatively analyzes the condition of the emergency patient in a high-dimensional feature space and dynamically optimizes the level of detail of product proposals using AI, thereby achieving remarkable effects such as improved information transmission efficiency, reduced risk of misunderstanding, reduced user burden, and improved response accuracy in emergencies. Specific application fields include severity-based product proposals at emergency sites, self-medication support in drugstore stores, family support in home care settings, and emergency response at event venues or public transportation.

The proposal unit can apply different proposal algorithms according to the category of the emergency patient when proposing a product. The proposal unit applies different proposal algorithms according to the category of the emergency patient when proposing a product. The category of the emergency patient may include, for example, type of illness and severity of symptoms, but is not limited thereto. For example, when the emergency patient is a child, the proposal unit applies a product proposal algorithm for children. When the emergency patient is an elderly person, the proposal unit may apply a product proposal algorithm for the elderly. Furthermore, when the emergency patient is an adult, the proposal unit may apply a product proposal algorithm for adults. By applying proposal algorithms according to the category of the emergency patient, appropriate product proposals can be made. Some or all of the above-described processing in the proposal unit may be performed using AI or without using AI. For example, the proposal unit may input category data of the emergency patient into generative AI and have the generative AI apply the proposal algorithm. Specifically, the proposal unit inputs attribute data such as age (e.g., 5 years, 75 years, 35 years), gender, underlying diseases, medical history, and symptom category (e.g., infectious disease, trauma, chronic disease) of the emergency patient as input data to the AI model. The AI model encodes the attribute data as a multidimensional feature vector (e.g., 64 dimensions) and outputs labels such as “child”, “elderly”, “adult” as a probability distribution (e.g., child 0.8, elderly 0.1, adult 0.1) in the category determination unit. Examples of input to the AI include “age: 4 years, symptom: fever” and “age: 80 years, symptom: fall”. Examples of AI output include “category: child 0.95” and “category: elderly 0.90”. The proposal unit determines the category score in the threshold determination unit and performs rule-based processing such as “if the child score is 0.7 or higher, apply the child product proposal algorithm (e.g., dosage adjustment, consideration of taste and shape, emphasis on allergy risk)”, “if the elderly score is 0.7 or higher, apply the elderly product proposal algorithm (e.g., ease of swallowing, risk of interactions, consideration of medication history)”, and “if the adult score is 0.7 or higher, apply the adult product proposal algorithm (e.g., standard dosage, emphasis on versatility)”. The product proposal itself uses the diagnosis result and user attributes as input, matches them with the product database (product name, ingredients, indications, inventory information, etc.), and outputs structured text such as recommended product name, recommendation reason, and recommendation score (e.g., “Recommended product: antipyretic for children, recommendation reason: based on age and symptoms”). In subsequent processing, the proposal unit sends these recommendation results and algorithm application parameters to the user interface unit and presents the product proposal in a form optimized for AR glasses, smartphones, or tablet screens. As a technical effect, the proposal unit, unlike conventional uniform product proposals or operations dependent on human subjective judgment, quantitatively analyzes the category of the emergency patient in a high-dimensional feature space and optimizes category-adaptive product proposal algorithms using AI, thereby achieving remarkable effects such as improved accuracy of product selection, reduced risk of incorrect selection, reduced user burden, and realization of individual optimization. Specific application fields include individualized product proposals in pediatrics, elderly care facilities, and general emergency sites, self-medication support in drugstore stores, family support in home care settings, and emergency response at event venues or public transportation.

The proposal unit can estimate the user's emotion and adjust the length of proposals based on the estimated emotion of the user. The proposal unit estimates the user's emotion and adjusts the length of proposals based on the estimated emotion of the user. Estimation of the user's emotion may be realized using an emotion engine or generative AI, such as an emotion estimation function. Generative AI may be a text-generating AI (e.g., LLM) or a multimodal generative AI, but is not limited thereto. For example, when the user is nervous, the proposal unit provides a short and concise proposal. When the user is relaxed, the proposal unit may provide a detailed proposal. Furthermore, when the user is in a hurry, the proposal unit may provide a prompt proposal. By adjusting the length of proposals according to the user's emotion, appropriate proposals can be made. Some or all of the above-described processing in the proposal unit may be performed using AI or without using AI. For example, the proposal unit may input emotion data of the user into generative AI and have the generative AI adjust the length of the proposal. Specifically, the proposal unit inputs the user's facial expression image (e.g., 1×3×224×224 RGB tensor), audio data (e.g., 5 seconds of audio waveform, 16 kHz sampling), and biometric sensor data (e.g., time-series vectors of heart rate, skin conductance, etc.) into a multimodal neural network for emotion estimation, which outputs emotion labels such as “nervous”, “relaxed”, “in a hurry” as a probability distribution. Examples of input to the AI include data such as “image of a tense face”, “fast speech audio”, and “heart rate above 100 bpm”, which result in a high nervousness score. Examples of AI output include “nervous: 0.85”, “relaxed: 0.10”, “in a hurry: 0.05”. The proposal unit determines these emotion scores in the threshold determination unit and performs rule-based processing such as “if the nervousness score is 0.7 or higher, display the product proposal text in 1-2 short sentences focusing only on key points”, “if the relaxation score is 0.7 or higher, display a long text with detailed product description, ingredient information, usage precautions, etc.”, and “if the in-a-hurry score is 0.7 or higher, display only the most important product name and reason in one sentence”. The product proposal itself uses the diagnosis result and user attributes as input, matches them with the product database (product name, ingredients, indications, inventory information, etc.), and outputs structured text such as recommended product name, recommendation reason, and recommendation score (e.g., “Recommended product: oral rehydration solution, recommendation reason: dehydration is strongly suspected, recommendation score: 0.92”). In subsequent processing, the proposal unit sends these recommendation results and length parameters to the user interface unit and presents the product proposal in a form optimized for AR glasses, smartphones, or tablet screens. As a technical effect, the proposal unit, unlike conventional uniform product proposal displays or operations dependent on human subjective judgment, quantitatively estimates the user's psychological state in a high-dimensional feature space and dynamically optimizes the length of product proposals using AI, thereby achieving remarkable effects such as improved information transmission efficiency, reduced user stress, reduced risk of misunderstanding, and improved response accuracy in emergencies. Specific application fields include stress response at emergency sites, self-medication support in drugstore stores, family support in home care settings, and emergency response at event venues or public transportation.

The proposal unit can determine the priority order of proposals based on the condition of the emergency patient when proposing a product. The proposal unit determines the priority order of proposals based on the condition of the emergency patient when proposing a product. The condition of the emergency patient may include, for example, vital signs and severity of symptoms, but is not limited thereto. For example, when the condition of the emergency patient is severe, the proposal unit prioritizes the most important products. When the condition of the emergency patient is mild, the proposal unit may prioritize general products. Furthermore, the proposal unit may prioritize optimal products according to the condition of the emergency patient. By determining the priority order of proposals according to the condition of the emergency patient, important products can be proposed preferentially. Some or all of the above-described processing in the proposal unit may be performed using AI or without using AI. For example, the proposal unit may input condition data of the emergency patient into generative AI and have the generative AI determine the priority order of proposals. Specifically, the proposal unit inputs vital signs of the emergency patient (e.g., numerical data such as heart rate, blood pressure, body temperature, respiratory rate), symptom scores (e.g., probability values such as “dehydration: 0.85”, “fever: 0.10”), and consciousness level (e.g., scores such as JCS, GCS) as input data to the AI model. The AI model encodes these condition data as a multidimensional feature vector (e.g., 128 dimensions) and outputs labels such as “severe”, “moderate”, “mild” as a probability distribution (e.g., severe 0.8, moderate 0.1, mild 0.1) in the severity determination unit. Examples of input to the AI include “heart rate 120 bpm, blood pressure 80/50 mmHg, consciousness level JCS3” and “body temperature 37.2° C., mild headache”. Examples of AI output include “severity: 0.90 (severe)” and “severity: 0.20 (mild)”. The proposal unit determines the severity score in the threshold determination unit and performs rule-based processing such as “if the severity score is 0.7 or higher, place highly urgent products (e.g., oral rehydration solution, emergency medicines) at the top of the product proposal list”, “if the mild score is 0.7 or higher, prioritize general products (e.g., over-the-counter antipyretics, analgesics)”. The product proposal itself uses the diagnosis result and user attributes as input, matches them with the product database (product name, ingredients, indications, inventory information, etc.), and outputs structured text such as recommended product name, recommendation reason, and recommendation score (e.g., “Recommended product: oral rehydration solution, recommendation reason: dehydration is strongly suspected, recommendation score: 0.92”). Examples of output include “Recommended product 1: oral rehydration solution (high severity), Recommended product 2: antipyretic (moderate severity)”. In subsequent processing, the proposal unit sends these recommendation results and priority parameters to the user interface unit and presents the product proposal in a form optimized for AR glasses, smartphones, or tablet screens. The user can select products or display additional information via voice commands or touchpad operations. As a technical effect, the proposal unit, unlike conventional uniform product proposal order or operations dependent on human subjective judgment, quantitatively analyzes the condition of the emergency patient in a high-dimensional feature space and dynamically optimizes the priority order of product proposals using AI, thereby achieving remarkable effects such as improved information transmission efficiency, reduced risk of incorrect selection, reduced user burden, and improved response accuracy in emergencies. Specific application fields include severity-based product proposals at emergency sites, self-medication support in drugstore stores, family support in home care settings, and emergency response at event venues or public transportation.

The proposal unit can adjust the order of proposals based on the relevance to the emergency patient when proposing a product. The proposal unit adjusts the order of proposals based on the relevance to the emergency patient when proposing a product. The relevance to the emergency patient may include, for example, severity of symptoms and medical history, but is not limited thereto. For example, the proposal unit first proposes products most relevant to the condition of the emergency patient. The proposal unit may then propose products in order of decreasing relevance to the condition of the emergency patient. Furthermore, the proposal unit may propose products in the optimal order based on the condition of the emergency patient. By adjusting the order of proposals based on relevance to the emergency patient, products can be proposed in an appropriate order. Some or all of the above-described processing in the proposal unit may be performed using AI or without using AI. For example, the proposal unit may input relevance data of the emergency patient into generative AI and have the generative AI adjust the order of proposals. Specifically, the proposal unit inputs symptom scores of the emergency patient (e.g., “dehydration: 0.85”, “fever: 0.10”), medical history (e.g., diabetes, hypertension, allergies), and recent treatment history (e.g., medications being taken, diseases under treatment) as input data to the AI model. The AI model encodes these data as a multidimensional feature vector (e.g., 128 dimensions), matches them with the product database (product name, ingredients, indications, contraindication information, etc.), and calculates a relevance score for each product (e.g., relevance to dehydration 0.92, relevance to fever 0.10). Examples of input to the AI include “symptom: dehydration, medical history: diabetes” and “symptom: fever, medical history: hypertension”. Examples of AI output include “Product A: relevance 0.95” and “Product B: relevance 0.60”. The proposal unit determines the relevance score in the threshold determination unit and rearranges the product proposal list in order of decreasing relevance. For example, “display products with relevance 0.9 or higher at the top”, “display products with relevance 0.5 to 0.9 next”, etc. The product proposal itself uses the diagnosis result and user attributes as input, and outputs structured text such as recommended product name, recommendation reason, and recommendation score (e.g., “Recommended product: oral rehydration solution, recommendation reason: high relevance to dehydration”). In subsequent processing, the proposal unit sends these recommendation results and order parameters to the user interface unit and presents the product proposal in a form optimized for AR glasses, smartphones, or tablet screens. The user can select products or display additional information via voice commands or touchpad operations. As a technical effect, the proposal unit, unlike conventional uniform product proposal order or operations dependent on human subjective judgment, quantitatively analyzes the condition and medical history of the emergency patient in a high-dimensional feature space and optimizes the order of product proposals based on relevance using AI, thereby achieving remarkable effects such as improved accuracy of product selection, reduced risk of incorrect selection, reduced user burden, and realization of individual optimization. Specific application fields include symptom-based product proposals at emergency sites, self-medication support in drugstore stores, family support in home care settings, and emergency response at event venues or public transportation.

The display unit can estimate the user's emotion and adjust the display method of procedures for first aid based on the estimated emotion of the user. The display unit estimates the user's emotion and adjusts the display method of procedures for first aid based on the estimated emotion of the user. Estimation of the user's emotion may be realized using an emotion engine or generative AI, such as an emotion estimation function. Generative AI may be a text-generating AI (e.g., LLM) or a multimodal generative AI, but is not limited thereto. For example, when the user is nervous, the display unit provides a simple and highly visible display method. When the user is relaxed, the display unit may provide a display method including detailed information. Furthermore, when the user is in a hurry, the display unit may provide a display method focusing on key points. By adjusting the display method according to the user's emotion, procedures for first aid can be displayed with high visibility. Some or all of the above-described processing in the display unit may be performed using AI or without using AI. For example, the display unit may input emotion data of the user into generative AI and have the generative AI adjust the display method. Specifically, the display unit inputs the user's facial expression image (e.g., 1×3×224×224 RGB tensor), audio data (e.g., 5 seconds of audio waveform, 16 kHz sampling), and biometric sensor data (e.g., time-series vectors of heart rate, skin conductance, etc.) into a multimodal neural network for emotion estimation, which outputs emotion labels such as “nervous”, “relaxed”, “in a hurry” as a probability distribution. Examples of input to the AI include data such as “image of a tense face”, “fast speech audio”, and “heart rate above 100 bpm”, which result in a high nervousness score. Examples of AI output include “nervous: 0.85”, “relaxed: 0.10”, “in a hurry: 0.05”. The display unit determines these emotion scores in the threshold determination unit and performs rule-based processing such as “if the nervousness score is 0.7 or higher, display procedures for first aid in large font, simple colors, and short sentences focusing only on key points”, “if the relaxation score is 0.7 or higher, display with detailed procedure explanations, illustrations, and supplementary information”, and “if the in-a-hurry score is 0.7 or higher, emphasize only the most important procedures”. In subsequent processing, the display unit sends these display method parameters to the user interface unit and presents procedures for first aid in a form optimized for AR glasses, smartphones, or tablet screens. As a technical effect, the display unit, unlike conventional uniform procedure displays or operations dependent on human subjective judgment, quantitatively estimates the user's psychological state in a high-dimensional feature space and dynamically optimizes the display of procedures for first aid using AI, thereby achieving remarkable effects such as improved visibility, improved information transmission efficiency, reduced user stress, and reduced risk of erroneous operation. Specific application fields include stress response at emergency sites, family support in home care settings, simulation training in medical education settings, and emergency response in public spaces.

The display unit can refer to the user's past first aid history when displaying procedures for first aid and select an optimal display method. The display unit refers to the user's past first aid history when displaying procedures for first aid and selects an optimal display method. The past first aid history may include, for example, records of first aid performed and success rates, but is not limited thereto. For example, the display unit may select a display method similar to that used for first aid methods the user has succeeded with in the past. The display unit may also avoid display methods used for first aid methods the user has failed with in the past and select a different display method. Furthermore, the display unit may select the most effective display method based on the user's past first aid history. By referring to past first aid history, the optimal display method can be selected. Some or all of the above-described processing in the display unit may be performed using AI or without using AI. For example, the display unit may input the user's past first aid history data into generative AI and have the generative AI select the optimal display method. Specifically, the display unit refers to a first aid history database accumulated for each user (e.g., procedure implementation date and time, success/failure flag, display method parameters, user reactions, etc. as structured data), and inputs these history data as time-series vectors (e.g., 128-dimensional feature vectors for each case) to the AI model. Examples of input data include “May 1, 2023: cardiopulmonary resuscitation, simple display, success” and “Jun. 10, 2023: hemostasis, detailed display, failure”. The display unit inputs these history data to a time-series analysis neural network based on LSTM or Transformer and trains the model on past success/failure patterns. The AI model calculates similarity to past history for new cases (e.g., current first aid type, user attributes, environmental conditions) and outputs optimal display parameters (e.g., font size, amount of information, presence/absence of illustrations). Examples of AI output include “Recommended display method: simple display, large font, no illustrations” and “Recommended display method: detailed display, with illustrations”. In subsequent processing, the display unit automatically sets display parameters in the user interface unit based on the AI model's output and presents guidance to the user such as “Procedures will be displayed in this format”. As a technical effect, the display unit, unlike conventional uniform procedure displays or user-initiated operations, analyzes individual users' past history in a high-dimensional feature space and optimizes personalized display of procedures for first aid using AI, thereby achieving remarkable effects such as improved information transmission efficiency, reduced risk of erroneous operation, and reduced user burden. Specific application fields include individualized procedure display support at emergency sites, utilization of family history in home care settings, display guidance based on training history in medical education settings, and feedback utilization of emergency response history in public spaces.

The display unit can customize the display content based on the current condition of the emergency patient when displaying procedures for first aid. The display unit customizes the display content based on the current condition of the emergency patient when displaying procedures for first aid. The current condition of the emergency patient may include, for example, vital signs and severity of symptoms, but is not limited thereto. For example, when the condition of the emergency patient is severe, the display unit displays detailed procedures for first aid. When the condition of the emergency patient is mild, the display unit may display concise procedures for first aid. Furthermore, the display unit may display optimal procedures for first aid according to the condition of the emergency patient. By customizing the display content based on the current condition of the emergency patient, appropriate procedures for first aid can be displayed. Some or all of the above-described processing in the display unit may be performed using AI or without using AI. For example, the display unit may input current condition data of the emergency patient into generative AI and have the generative AI customize the display content. Specifically, the display unit inputs vital signs of the emergency patient (e.g., numerical data such as heart rate, blood pressure, body temperature, respiratory rate), symptom scores (e.g., “dehydration: 0.85”, “fever: 0.10”), and consciousness level (e.g., scores such as JCS, GCS) as input data to the AI model. The AI model encodes these condition data as a multidimensional feature vector (e.g., 128 dimensions) and outputs labels such as “severe”, “moderate”, “mild” as a probability distribution (e.g., severe 0.8, moderate 0.1, mild 0.1) in the severity determination unit. Examples of input to the AI include “heart rate 120 bpm, blood pressure 80/50 mmHg, consciousness level JCS3” and “body temperature 37.2° C., mild headache”. Examples of AI output include “severity: 0.90 (severe)” and “severity: 0.20 (mild)”. The display unit determines the severity score in the threshold determination unit and performs rule-based processing such as “if the severity score is 0.7 or higher, display procedures for first aid in detail (e.g., all steps, precautions, with illustrations)”, “if the mild score is 0.7 or higher, display only key points concisely”. In subsequent processing, the display unit sends these display content parameters to the user interface unit and presents procedures for first aid in a form optimized for AR glasses, smartphones, or tablet screens. As a technical effect, the display unit, unlike conventional uniform procedure displays or operations dependent on human subjective judgment, quantitatively analyzes the condition of the emergency patient in a high-dimensional feature space and dynamically optimizes the display content of procedures for first aid using AI, thereby achieving remarkable effects such as improved information transmission efficiency, reduced risk of erroneous operation, reduced user burden, and improved response accuracy in emergencies. Specific application fields include severity-based procedure displays at emergency sites, family support in home care settings, simulation training in medical education settings, and emergency response in public spaces.

The display unit can estimate the user's emotion and adjust the display order of procedures for first aid based on the estimated emotion of the user. The display unit estimates the user's emotion and adjusts the display order of procedures for first aid based on the estimated emotion of the user. Estimation of the user's emotion may be realized using an emotion engine or generative AI, such as an emotion estimation function. Generative AI may be a text-generating AI (e.g., LLM) or a multimodal generative AI, but is not limited thereto. For example, when the user is nervous, the display unit displays the most important procedures first. When the user is relaxed, the display unit may display all procedures in order. Furthermore, when the user is in a hurry, the display unit may display key procedures first. By adjusting the display order according to the user's emotion, the most important procedures can be displayed first. Some or all of the above-described processing in the display unit may be performed using AI or without using AI. For example, the display unit may input emotion data of the user into generative AI and have the generative AI adjust the display order. Specifically, the display unit inputs the user's facial expression image (e.g., 1×3×224×224 RGB tensor), audio data (e.g., 5 seconds of audio waveform, 16 kHz sampling), and biometric sensor data (e.g., time-series vectors of heart rate, skin conductance, etc.) into a multimodal neural network for emotion estimation, which outputs emotion labels such as “nervous”, “relaxed”, “in a hurry” as a probability distribution. Examples of input to the AI include data such as “image of a tense face”, “fast speech audio”, and “heart rate above 100 bpm”, which result in a high nervousness score. Examples of AI output include “nervous: 0.85”, “relaxed: 0.10”, “in a hurry: 0.05”. The display unit determines these emotion scores in the threshold determination unit and performs rule-based processing such as “if the nervousness score is 0.7 or higher, place the most important procedures (e.g., chest compressions, hemostasis) at the top of the procedure list”, “if the relaxation score is 0.7 or higher, display all procedures in standard order”, and “if the in-a-hurry score is 0.7 or higher, display only key procedures at the top”. In subsequent processing, the display unit sends these display order parameters to the user interface unit and presents procedures for first aid in a form optimized for AR glasses, smartphones, or tablet screens. As a technical effect, the display unit, unlike conventional uniform procedure display order or operations dependent on human subjective judgment, quantitatively estimates the user's psychological state in a high-dimensional feature space and dynamically optimizes the display order of procedures for first aid using AI, thereby achieving remarkable effects such as prevention of overlooking important procedures, improved information transmission efficiency, reduced user stress, and reduced risk of erroneous operation. Specific application fields include stress response at emergency sites, family support in home care settings, simulation training in medical education settings, and emergency response in public spaces.

The display unit can select an optimal display method by considering the user's device information when displaying procedures for first aid. The display unit selects an optimal display method by considering the user's device information when displaying procedures for first aid. Device information may include, for example, device type, screen size, and resolution, but is not limited thereto. For example, when the user is using a smartphone, the display unit provides a display method adapted to the screen size. When the user is using a tablet, the display unit may provide a display method optimized for a large screen. Furthermore, when the user is using a smartwatch, the display unit may provide a concise and highly visible display method. By considering device information, the optimal display method can be selected. Some or all of the above-described processing in the display unit may be performed using AI or without using AI. For example, the display unit may input the user's device information into generative AI and have the generative AI select the optimal display method. Specifically, the display unit inputs device information such as device type (e.g., smartphone, tablet, AR glasses, smartwatch), screen size (e.g., 5.5 inches, 10 inches, 1.5 inches), and resolution (e.g., 1920×1080, 2560×1600, 320×320) as input data to the AI model. The AI model encodes these device information as a multidimensional feature vector (e.g., 32 dimensions) and outputs optimal display parameters (e.g., font size, amount of information, layout, presence/absence of illustrations). Examples of input to the AI include “device: smartphone, screen size 5.5 inches” and “device: smartwatch, screen size 1.5 inches”. Examples of AI output include “Recommended display method: large font, less information, vertical scroll” and “Recommended display method: illustration-focused, horizontal scroll”. In subsequent processing, the display unit automatically sets display parameters in the user interface unit based on the AI model's output and presents guidance to the user such as “Procedures will be displayed optimized for this device”. As a technical effect, the display unit, unlike conventional uniform procedure displays or user-initiated operations, analyzes device information in a high-dimensional feature space and optimizes device-adaptive display of procedures for first aid using AI, thereby achieving remarkable effects such as improved visibility, improved information transmission efficiency, reduced user burden, and reduced risk of erroneous operation. Specific application fields include support for various devices at emergency sites, family support in home care settings, device-specific training in medical education settings, and emergency response in public spaces.

The display unit can refer to related literature of the emergency patient when displaying procedures for first aid to improve the display content. The display unit refers to related literature of the emergency patient when displaying procedures for first aid to improve the display content. Related literature may include, for example, medical papers and guidelines, but is not limited thereto. For example, the display unit refers to the latest research papers on the condition of the emergency patient to improve the display content of procedures for first aid. The display unit may also refer to past literature on the symptoms of the emergency patient to improve the display content of procedures for first aid. Furthermore, the display unit may refer to literature on the treatment of the emergency patient to improve the display content of procedures for first aid. By referring to related literature, the display content can be improved. Some or all of the above-described processing in the display unit may be performed using AI or without using AI. For example, the display unit may input related literature data of the emergency patient into generative AI and have the generative AI improve the display content. Specifically, the display unit obtains text data (e.g., paper titles, abstracts, main text, figure captions) from medical literature databases (e.g., guideline collections, case report collections), vectorizes them using a natural language processing module (e.g., BERT-based text encoder), and inputs them together with condition data of the emergency patient (e.g., symptom scores, vital signs) to the AI model. The AI model integrates the literature information and condition data as a multidimensional feature vector (e.g., 256 dimensions) and outputs parameters for optimizing the display content of procedures for first aid (e.g., latest recommended procedures, precautions, reference literature links). Examples of input to the AI include “symptom: rash, literature: guidelines for first aid in case of rash” and “symptom: impaired consciousness, literature: latest paper on emergency resuscitation”. Examples of AI output include “Recommended procedure: compliant with latest guidelines” and “Reference literature link: paper published in 2023”. In subsequent processing, the display unit automatically sets display content parameters in the user interface unit based on the AI model's output and presents guidance to the user such as “Procedures will be displayed based on the latest medical knowledge”. As a technical effect, the display unit, unlike conventional paper manuals or procedure displays dependent on human experience, integratively analyzes the latest medical knowledge in a high-dimensional feature space and optimizes knowledge-extended display of procedures for first aid using AI, thereby achieving remarkable effects such as reduced risk of erroneous operation, improved information transmission efficiency, reduced user burden, and improved medical safety. Specific application fields include response to emerging diseases at emergency sites, reflection of latest guidelines in home care settings, case-based training in medical settings, and emergency response in public spaces.

The system according to the embodiment is not limited to the above examples and may be variously modified as follows, for example. Specifically, by changing the architecture of the AI model, the system may use not only convolutional neural networks (CNN) but also Vision Transformers or self-supervised learning models in the image analysis unit. In the audio analysis unit, in addition to acoustic feature extraction, RNN or Transformer-based models for speech emotion recognition and audio event detection may be combined. Furthermore, in the product proposal unit, not only a single large language model but also a multi-model configuration linking a symptom classification dedicated model and a product recommendation dedicated model, or reinforcement learning for optimizing the recommendation algorithm, may be introduced. From the perspective of data flow, by combining distributed databases in the cloud and real-time inference on edge devices, communication load can be reduced and response speed improved. Additionally, in the user interface unit, an AI module that automatically generates display layouts and operation methods optimized for various devices such as AR glasses, smartphones, tablets, and smartwatches may be added. As learning methods for the AI model, transfer learning, self-distillation, and data augmentation (e.g., image rotation, noise addition, audio pitch conversion) may be applied to achieve highly accurate inference even in environments with small amounts of data. With these variations, the system can flexibly adapt to various field environments and user attributes, such as emergency sites, home care, medical education, public spaces, and event venues, and achieve overall improvement in diagnostic accuracy, product recommendation accuracy, first aid guide accuracy, and user experience. As a technical effect, the flexibility, scalability, and field adaptability of the system are dramatically improved, and compared to conventional fixed AI systems or human-dependent operations, remarkable effects such as reduced operational costs, reduced risk of misdiagnosis or erroneous operation, and improved user satisfaction are achieved.

The acquisition unit can analyze the user's past emergency patient response history when acquiring an image of an emergency patient and select an optimal image acquisition method. For example, the acquisition unit may select an image acquisition method similar to that used in successful emergency patient responses by the user in the past. The acquisition unit may also avoid image acquisition methods used in failed emergency patient responses by the user in the past and select a different image acquisition method. Furthermore, the acquisition unit may select the most effective image acquisition method based on the user's past response history. By analyzing past response history, the optimal image acquisition method can be selected. Specifically, the acquisition unit refers to an emergency patient response history database accumulated for each user (e.g., date and time of first aid performed, diagnosis result, camera settings at the time of image acquisition, environmental conditions, success/failure flag, etc. as structured data), and inputs these history data as time-series vectors (e.g., 128-dimensional feature vectors for each response case) to the AI model. Examples of input data include “May 1, 2023: outdoors, clear weather, camera setting ISO400, success” and “Jun. 10, 2023: indoors, dim lighting, camera setting ISO800, failure”. The acquisition unit inputs these history data to a time-series analysis neural network based on LSTM or Transformer and trains the model on past success/failure patterns. The AI model calculates similarity to past history for new cases (e.g., current environment, user attributes, condition of the emergency patient) and outputs optimal image acquisition parameters (e.g., camera angle, exposure, whether to use burst mode, etc.). Examples of AI output include “Recommended camera setting: ISO400, shutter speed 1/60, prioritize close-up of face” and “Recommended acquisition method: two consecutive shots of full body and close-up of face”. In subsequent processing, the acquisition unit automatically sets parameters in the camera control module based on the AI model's output and presents guidance to the user such as “Please take a picture from this angle”. As a technical effect, the acquisition unit, unlike conventional uniform image acquisition procedures or user-initiated operations, analyzes individual users' past history in a high-dimensional feature space and optimizes personalized image acquisition using AI, thereby achieving remarkable effects such as improved image quality, improved diagnostic accuracy, reduced user burden, and reduced risk of erroneous operation. Specific application fields include individualized image acquisition support at emergency sites, utilization of family response history in home care settings, shooting guidance based on training history in medical education settings, and feedback utilization of emergency response history in public spaces.

The acquisition unit can perform filtering based on the user's current location information or environment when acquiring an image of an emergency patient. For example, when the user is outdoors, the acquisition unit acquires images considering the ambient light. When the user is indoors, the acquisition unit may acquire images considering the lighting conditions. Furthermore, the acquisition unit may select an optimal image acquisition method based on the user's location information. By performing filtering based on location information or environment, appropriate images can be acquired. Specifically, the acquisition unit collects location information (e.g., latitude, longitude, altitude, indoor/outdoor determination, such as latitude 35.6895, longitude 139.6917, outdoor flag 1) obtained from GPS modules, Wi-Fi positioning, Bluetooth beacons, etc., and multidimensional environmental data obtained from environmental sensors, such as temperature (e.g., 28.5° C.), humidity (e.g., 60%), illuminance (e.g., 500 lx), and noise level (e.g., 65 dB), in real time. The acquisition unit inputs these location and environmental data as a 128-dimensional feature vector to the AI model and infers optimal filtering conditions for image acquisition (e.g., exposure compensation, automatic white balance adjustment, noise reduction strength). Examples of input to the AI include “outdoors, clear weather, illuminance 2000 lx” and “indoors, fluorescent light, illuminance 300 lx, noise 80 dB”. Examples of AI output include “Exposure +1.0, ISO200, strong noise reduction” and “White balance: fluorescent light mode”. In subsequent processing, the acquisition unit automatically sets parameters in the camera control module based on the AI model's output and presents guidance to the user such as “Adjust the angle due to backlight”. As a technical effect, the acquisition unit, unlike conventional uniform camera settings or user-initiated operations, analyzes real-time location and environmental data in a high-dimensional feature space and dynamically optimizes image acquisition using AI, thereby achieving remarkable effects such as improved image quality, improved diagnostic accuracy, reduced user burden, and reduced risk of erroneous shooting. Specific application fields include emergency response at outdoor event venues, adaptation to environmental changes in home care settings, optimization of image acquisition under various lighting conditions in medical settings, and response to noisy environments in public transportation.

The acquisition unit can analyze audio information around the user when acquiring an image of an emergency patient and preferentially acquire related images. For example, the acquisition unit may detect the emergency patient's breathing sounds from surrounding audio and preferentially acquire images of that part. The acquisition unit may also detect the emergency patient's voice from surrounding audio and preferentially acquire images of that part. Furthermore, the acquisition unit may analyze environmental sounds from surrounding audio and preferentially acquire images related to the condition of the emergency patient. By analyzing audio information around the user, related images can be preferentially acquired. Specifically, the acquisition unit acquires audio data (e.g., 16 kHz sampling, 5 seconds of audio waveform) in real time from microphones installed in AR glasses, smartphones, etc., and vectorizes it using an audio recognition/acoustic feature extraction module (e.g., MFCC extraction, spectrogram generation). The acquisition unit inputs these audio feature vectors to a neural network for audio event detection (e.g., CNN+RNN hybrid model), which outputs audio event labels such as “breathing sound”, “groaning”, “environmental noise” as a probability distribution (e.g., breathing sound 0.8, groaning 0.6, noise 0.2). Examples of input to the AI include “audio waveform with periodic detection of breathing sounds” and “audio with mixed groaning”. Examples of AI output include “breathing sound: 0.85”, “groaning: 0.60”, “environmental noise: 0.10”. The acquisition unit determines these audio event scores in the threshold determination unit and performs rule-based processing such as “if the breathing sound score is 0.7 or higher, preferentially acquire chest images”, “if the groaning score is 0.5 or higher, preferentially acquire face/mouth images”. In subsequent processing, the image acquisition priority list (e.g., chest>face>whole body) is sent to the camera control module, and guidance such as “Please take a picture of the chest” is displayed to the user. As a technical effect, the acquisition unit, unlike conventional image acquisition using only visual information, analyzes audio information in a high-dimensional feature space and realizes multimodal image acquisition priority control using AI, thereby achieving remarkable effects such as prevention of missing important areas, improved diagnostic accuracy, reduced user burden, and reduced risk of erroneous operation. Specific application fields include support for detection of abnormal breathing at emergency sites, audio event-linked shooting in home care settings, simulation training with audio linkage in medical education settings, and emergency response in public spaces.

The acquisition unit can analyze the user's online activity when acquiring an image of an emergency patient and acquire related images. For example, the acquisition unit may obtain information about the condition of the emergency patient from the user's social media posts and preferentially acquire related images. The acquisition unit may also acquire images of the emergency patient based on location information from the user's social media. Furthermore, the acquisition unit may analyze the user's social media friend relationships and acquire images related to the emergency patient. By analyzing social media activity, related images can be acquired. Specifically, the acquisition unit collects the user's social media post data (e.g., post text, images, location information, post time, friend tags as structured data) and browsing history (e.g., page URLs viewed, search keywords, access times), and vectorizes them using a natural language processing module (e.g., BERT-based text encoder) and a graph neural network (for friend relationship network analysis). The acquisition unit inputs these online activity feature vectors to the AI model, which outputs the condition of the emergency patient and relevance scores (e.g., match between post content and emergency patient symptoms, match of location information, relevance of friend relationships). Examples of input to the AI include “post text: ‘My friend collapsed’”, “location information: Shinjuku Station”, “friend tag: Mr. A”. Examples of AI output include “relevance score: 0.92 (high probability that friend Mr. A is the emergency patient)”, “location match: 0.85”. The acquisition unit determines these relevance scores in the threshold determination unit and performs rule-based processing such as “if the relevance score is 0.8 or higher, preferentially acquire face images of the relevant person”, “if the location match is 0.7 or higher, acquire images around the site”. In subsequent processing, the image acquisition priority list is sent to the camera control module, and guidance such as “Please take a picture of Mr. A's face” is displayed to the user. As a technical effect, the acquisition unit, unlike conventional image acquisition using only on-site information, analyzes online activity data in a high-dimensional feature space and realizes multi-source collaborative image acquisition optimization using AI, thereby achieving remarkable effects such as improved accuracy in identifying relevant persons and sites, improved diagnostic accuracy, reduced user burden, and reduced risk of erroneous shooting. Specific application fields include SNS-linked emergency response at event venues, utilization of family networks in home care settings, patient history linkage in medical settings, and identification of emergency patients in crowds in public spaces.

The analysis unit can refer to past health data of the emergency patient during image analysis to improve the accuracy of the analysis. For example, the analysis unit may refer to past health checkup data of the emergency patient to improve the accuracy of image analysis. The analysis unit may also refer to past medical history of the emergency patient to improve the accuracy of image analysis. Furthermore, the analysis unit may refer to past treatment history of the emergency patient to improve the accuracy of image analysis. By referring to past health data, the accuracy of image analysis is improved. Specifically, the analysis unit obtains electronic medical record data of the emergency patient (e.g., diagnosis name, past illnesses, medication history, allergy information, time-series test values), health checkup results (e.g., blood test values, ECG waveforms, image diagnosis results), and treatment history (e.g., surgical history, hospitalization history, treatment responsiveness) from a structured database, and inputs these as feature vectors (e.g., 128-dimensional numerical vectors for each item) to the AI model. The analysis unit simultaneously inputs image data (e.g., 1×3×224×224 RGB tensor) and health data vectors to a multimodal neural network (e.g., ResNet-50 for image input, MLP for health data input, Transformer encoder for integration), which outputs the condition of the emergency patient (e.g., dehydration, fever, impaired consciousness, trauma) as a probability distribution (e.g., scores from 0.0 to 1.0 for each symptom). Examples of input to the AI include “image: pale complexion, supine posture”, “health data: diabetes in the past, recent HbA1c 8.2%”, “treatment history: under treatment for hypertension”. Examples of AI output include “dehydration: 0.85”, “fever: 0.10”, “impaired consciousness: 0.30”. The analysis unit determines these scores in the threshold determination unit and sends the diagnosis result to the product proposal unit or first aid guide unit. For training the AI model, pairs of image, health data, and diagnosis labels are used, and cross-entropy loss functions and multitask learning are applied. As a technical effect, the analysis unit, unlike conventional image-only analysis or diagnosis dependent on human experience, integratively analyzes past health data in a high-dimensional feature space and realizes individually optimized improvement of diagnostic accuracy using AI, thereby achieving remarkable effects such as reduced risk of misdiagnosis, improved diagnostic accuracy, reduced user burden, and improved medical safety. Specific application fields include diagnosis considering medical history at emergency sites, utilization of family history in home care settings, electronic medical record-linked diagnosis in medical settings, and emergency response in public spaces.

The acquisition unit can estimate a user's emotion and adjust the timing of acquiring the image of the emergency patient based on the estimated emotion of the user. For example, when the user is nervous, the acquisition unit delays image acquisition and waits for the user to calm down. When the user is in a hurry, the acquisition unit may acquire images quickly and start analysis. Furthermore, when the user is calm, the acquisition unit may adjust to acquire images at the optimal timing. By adjusting the timing of image acquisition according to the user's emotion, images can be acquired at the appropriate timing. Specifically, the acquisition unit simultaneously acquires the user's facial expression image (e.g., 1×3×224×224 RGB tensor), audio data (e.g., 5 seconds of audio waveform, 16 kHz sampling), and biometric sensor data (e.g., time-series vectors of heart rate, skin conductance, etc.), and inputs them to a multimodal neural network for emotion estimation (e.g., a model combining CNN for image input, RNN for audio input, and MLP for sensor input). The model integrates feature vectors extracted from each modality and outputs emotion labels such as “nervous”, “in a hurry”, “calm” as a probability distribution (e.g., nervous 0.7, in a hurry 0.2, calm 0.1). Examples of input include “image of a tense face”, “fast speech audio”, and “heart rate above 100 bpm”, which result in a high nervousness score. Examples of output include “nervous: 0.85”, “in a hurry: 0.10”, “calm: 0.05”. The acquisition unit determines these emotion scores in the threshold determination unit and performs rule-based processing such as “if the nervousness score is 0.7 or higher, delay image acquisition by 5 seconds”, “if the in-a-hurry score is 0.5 or higher, acquire immediately”, “if the calm score is 0.7 or higher, acquire at standard timing”. Furthermore, adjustment of image acquisition timing is sequentially re-evaluated in real time while monitoring changes in the user's state, so that optimal image data can be secured while minimizing the user's psychological burden. For training the AI model, a large-scale dataset with emotion labels (e.g., pairs of facial expressions, audio, biometric signals, and emotion labels) is used, and weights are optimized with a cross-entropy loss function. As a technical effect, the acquisition unit, unlike conventional image acquisition by simple timers or user-initiated operations, quantitatively estimates the user's psychological state in a high-dimensional feature space and realizes dynamic timing control using AI, thereby achieving remarkable effects such as improved accuracy of image acquisition, reduced user stress, and reduced risk of erroneous operation. Specific application fields include stress response at emergency sites, family support in home care settings, simulation training in medical education settings, and emergency response in public spaces.

The analysis unit can estimate a user's emotion and adjust the accuracy of image analysis based on the estimated emotion of the user. For example, when the user is nervous, the analysis unit increases the accuracy of image analysis and provides diagnosis results quickly. When the user is relaxed, the analysis unit may perform image analysis with normal accuracy. Furthermore, when the user is in a hurry, the analysis unit may increase the accuracy of image analysis and provide diagnosis results quickly. By adjusting the accuracy of image analysis according to the user's emotion, diagnosis results can be provided quickly. Specifically, the analysis unit simultaneously acquires facial expression images (e.g., 1×3×224×224 RGB tensor), audio data (e.g., 5 seconds of audio waveform, 16 kHz sampling), and biometric sensor data (e.g., time-series vectors of heart rate, skin conductance, etc.) for emotion estimation, and inputs them to a multimodal neural network for emotion estimation (a model combining CNN for image input, RNN for audio input, and MLP for sensor input). The model integrates feature vectors extracted from each modality and outputs emotion labels such as “nervous”, “in a hurry”, “calm” as a probability distribution (e.g., nervous 0.7, in a hurry 0.2, calm 0.1). Examples of input include “image of a tense face”, “fast speech audio”, and “heart rate above 100 bpm”, which result in a high nervousness score. Examples of output include “nervous: 0.85”, “in a hurry: 0.10”, “calm: 0.05”. The analysis unit determines these emotion scores in the threshold determination unit and performs rule-based processing such as “if the nervousness score is 0.7 or higher, automatically set the accuracy parameters for image analysis (e.g., threshold for object detection model, number of ensemble inferences, strength of image preprocessing, etc.) to high-accuracy mode”, “if the calm score is 0.7 or higher, analyze in standard mode”. Image analysis itself is performed by combining, for example, feature extraction using CNNs such as ResNet-50 or EfficientNet, object detection using YOLOv8, segmentation using U-Net, and multimodal integration models based on Transformers. Examples of input to the AI include simultaneous input of image tensors such as “pale complexion, distressed expression, supine posture” and speech recognition text such as “collapsed”, “unconscious”. Examples of AI output include scores such as “dehydration: 0.85”, “fever: 0.10”, “trauma: 0.05”. For accuracy adjustment, parameters such as increasing the number of inferences for ensemble averaging, increasing the strength of noise removal in image preprocessing, and tightening thresholds are automatically controlled. In subsequent processing, the diagnosis result is sent to the product proposal unit or first aid guide unit, and optimal information is presented according to the user's situation. As a technical effect, the analysis unit, unlike conventional uniform image analysis accuracy settings or operations dependent on human subjective judgment, quantitatively estimates the user's psychological state in a high-dimensional feature space and dynamically controls the accuracy of image analysis using AI, thereby achieving remarkable effects such as improved diagnostic accuracy, reduced risk of misdiagnosis, reduced user stress, and improved responsiveness of the entire system. Specific application fields include stress response at emergency sites, family support in home care settings, simulation training in medical education settings, and emergency response in public spaces.

The proposal unit can estimate a user's emotion and adjust a method of expressing product proposals based on the estimated emotion of the user. For example, when the user is nervous, the proposal unit provides a simple and highly visible product proposal. When the user is relaxed, the proposal unit may provide a product proposal including detailed information. Furthermore, when the user is in a hurry, the proposal unit may provide a product proposal focusing on key points. By adjusting the method of expression according to the user's emotion, product proposals with high visibility can be provided. Specifically, the proposal unit simultaneously acquires facial expression images (e.g., 1×3×224×224 RGB tensor), audio data (e.g., 5 seconds of audio waveform, 16 kHz sampling), and biometric sensor data (e.g., time-series vectors of heart rate, skin conductance, etc.) for emotion estimation, and inputs them to a multimodal neural network for emotion estimation (a model combining CNN for image input, RNN for audio input, and MLP for sensor input). The model integrates feature vectors extracted from each modality and outputs emotion labels such as “nervous”, “relaxed”, “in a hurry” as a probability distribution (e.g., nervous 0.7, relaxed 0.2, in a hurry 0.1). Examples of input to the AI include “image of a tense face”, “fast speech audio”, and “heart rate above 100 bpm”, which result in a high nervousness score. Examples of AI output include “nervous: 0.85”, “relaxed: 0.10”, “in a hurry: 0.05”. The proposal unit determines these emotion scores in the threshold determination unit and performs rule-based processing such as “if the nervousness score is 0.7 or higher, display the product proposal text in large font, simple colors, and short sentences focusing only on key points”, “if the relaxation score is 0.7 or higher, display with detailed product description, ingredient information, usage precautions, etc.”, and “if the in-a-hurry score is 0.7 or higher, emphasize only the most important product name and reason”. The product proposal itself uses the diagnosis result and user attributes as input, matches them with the product database (product name, ingredients, indications, inventory information, etc.), and outputs structured text such as recommended product name, recommendation reason, and recommendation score (e.g., “Recommended product: oral rehydration solution, recommendation reason: dehydration is strongly suspected, recommendation score: 0.92”). In subsequent processing, the proposal unit sends these recommendation results and expression method parameters to the user interface unit and presents the product proposal in a form optimized for AR glasses, smartphones, or tablet screens. The user can select products or display additional information via voice commands or touchpad operations. As a technical effect, the proposal unit, unlike conventional uniform product proposal displays or operations dependent on human subjective judgment, quantitatively estimates the user's psychological state in a high-dimensional feature space and dynamically optimizes the method of expressing product proposals using AI, thereby achieving remarkable effects such as improved visibility, improved information transmission efficiency, reduced user stress, and reduced risk of misunderstanding. Specific application fields include stress response at emergency sites, self-medication support in drugstore stores, family support in home care settings, and emergency response at event venues or public transportation.

The proposal unit is capable of estimating a user's emotion and adjusting the length of proposals based on the estimated emotion of the user. For example, when the user is tense, the proposal unit provides a short and concise proposal. When the user is relaxed, the proposal unit can provide a detailed proposal. Furthermore, when the user is in a hurry, the proposal unit can provide a prompt proposal. By adjusting the length of proposals according to the user's emotion, appropriate proposals can be made. Specifically, the proposal unit inputs the user's facial expression image (e.g., 1×3×224×224 RGB tensor), audio data (e.g., 5 seconds of audio waveform, 16kHz sampling), and biometric sensor data (e.g., time-series vectors of heart rate, skin conductance, etc.) into a multimodal neural network for emotion estimation, and outputs emotion labels such as “tense,” “relaxed,” and “in a hurry” as a probability distribution. For example, when data such as an image of a stiff face, fast speech, and a heart rate of 100 bpm or higher are input to the AI, a high tension score is output. Example AI outputs include “tense: 0.85,” “relaxed: 0.10,” and “in a hurry: 0.05.” The proposal unit determines these emotion scores using a threshold determination unit, and performs rule-based processing such as “if the tension score is 0.7 or higher, display only the key points of the product proposal in 1-2 short sentences,” “if the relaxation score is 0.7 or higher, display a detailed product description, ingredient information, and usage precautions in a long sentence,” and “if the in-a-hurry score is 0.7 or higher, display only the most important product name and reason in one sentence.” The product proposal itself is generated by inputting the diagnosis result and user attributes, matching them with a product database (product name, ingredients, indications, inventory information, etc.), and outputting structured text such as recommended product name, recommended reason, and recommendation score (e.g., “Recommended product: oral rehydration solution, recommended reason: strong suspicion of dehydration, recommendation score: 0.92”). In subsequent processing, the proposal unit transmits these recommendation results and length parameters to the user interface unit, and presents the product proposal in a form optimized for the display of AR glasses, smartphones, or tablets. As a technical effect, the proposal unit, unlike conventional uniform product proposal displays or operations dependent on human subjective judgment, quantitatively estimates the user's psychological state in a high-dimensional feature space and dynamically optimizes the length of product proposals using AI, thereby achieving remarkable effects such as improved information transmission efficiency, reduced user stress, reduced risk of misunderstanding, and improved response accuracy in emergencies. Specific application fields include stress response in emergency situations, self-medication support in drugstore stores, family support in home care settings, and emergency response at event venues or on public transportation.

The display unit is capable of estimating a user's emotion and adjusting the display order of procedures for first aid based on the estimated emotion of the user. For example, when the user is tense, the display unit displays the most important procedures first. When the user is relaxed, the display unit can display the entire procedure in the standard order. Furthermore, when the user is in a hurry, the display unit can display the key procedures first. By adjusting the display order according to the user's emotion, the most important procedures can be displayed first. Specifically, the display unit inputs the user's facial expression image (e.g., 1×3×224×224 RGB tensor), audio data (e.g., 5 seconds of audio waveform, 16kHz sampling), and biometric sensor data (e.g., time-series vectors of heart rate, skin conductance, etc.) into a multimodal neural network for emotion estimation, and outputs emotion labels such as “tense,” “relaxed,” and “in a hurry” as a probability distribution. For example, when data such as an image of a stiff face, fast speech, and a heart rate of 100 bpm or higher are input to the AI, a high tension score is output. Example AI outputs include “tense: 0.85,” “relaxed: 0.10,” and “in a hurry: 0.05.” The display unit determines these emotion scores using a threshold determination unit, and performs rule-based processing such as “if the tension score is 0.7 or higher, place the most important procedures (e.g., chest compressions, hemostasis, etc.) at the top of the first aid procedure list,” “if the relaxation score is 0.7 or higher, display the entire procedure in the standard order,” and “if the in-a-hurry score is 0.7 or higher, display only the key procedures at the top.” In subsequent processing, the display unit transmits these display order parameters to the user interface unit, and presents the procedures for first aid in a form optimized for the display of AR glasses, smartphones, or tablets. As a technical effect, the display unit, unlike conventional uniform procedure display orders or operations dependent on human subjective judgment, quantitatively estimates the user's psychological state in a high-dimensional feature space and dynamically optimizes the display order of procedures for first aid using AI, thereby achieving remarkable effects such as prevention of overlooking important procedures, improved information transmission efficiency, reduced user stress, and reduced risk of operational errors. Specific application fields include stress response in emergency situations, family support in home care settings, simulation training in medical education settings, and emergency response in public spaces.

Below, the processing flow of Example of the Embodiment is briefly described. Specifically, the system operates by linking AI models with various sensors, devices, and databases at each step. In Step 1, the acquisition unit uses camera modules of AR glasses, smartphones, tablets, etc., to acquire real-time images of the entire body and close-up images of the face of the emergency patient, as well as continuous images from multiple angles (video streams, e.g., 1920×1080 pixels, 30 fps). The acquisition unit transmits the image data to a preprocessing unit, which performs noise removal, contrast adjustment, and segmentation of face and limb regions (e.g., using object detection models such as YOLOv8 or U-Net). The preprocessed images are numerically converted by a feature extraction unit into multidimensional feature vectors (e.g., 512 dimensions) such as facial color (RGB histogram, complexion estimation), facial expression (facial muscle patterns, action units), and posture (landmark extraction by skeleton estimation algorithms). In Step 2, the analysis unit inputs these feature vectors into a multimodal neural network (e.g., a model combining a ResNet-50 image input unit and a Transformer-based text encoder) and outputs the condition of the emergency patient (e.g., dehydration, fever, impaired consciousness, trauma, etc.) as a probability distribution (e.g., a score of 0.0 to 1.0 for each symptom). For example, image tensors of “pale facial color, agonized expression, supine posture” and speech recognition text such as “collapsed,” “unconscious,” etc., can be input simultaneously to the AI. Example AI outputs include “dehydration: 0.85,” “fever: 0.10,” “trauma: 0.05.” The analysis unit determines these scores using a threshold determination unit and performs rule-based processing such as “if the dehydration score is 0.7 or higher, recommend a beverage for hydration,” “if the fever score is 0.5 or higher, recommend an antipyretic,” etc. In Step 3, the proposal unit matches the diagnosis result with the drugstore product database (structured data such as product name, ingredients, indications, inventory information, etc.) and generates an optimal product candidate list (e.g., oral rehydration solution, antipyretic analgesics, cooling sheets, etc.). The product proposal unit inputs the diagnosis result and user attributes (age, gender, medical history, etc.) into a generative AI (e.g., LLM-based recommendation model) and can generate recommendation sentences with explanations such as “Recommended product: oral rehydration solution, reason: strong dehydration symptoms, rapid hydration required.” Example outputs include “Recommended product: oral rehydration solution, recommended reason: strong suspicion of dehydration based on facial color and posture” and “Recommended product: antipyretic, recommended reason: high fever score.” These recommendation results are overlay-displayed on the AR glasses display, and the user can select products or display additional information via voice commands or touchpad operations. As a technical effect, the system, unlike conventional responses dependent on human visual judgment or empirical rules, integrally analyzes diverse data such as images, audio, and text in a high-dimensional space and realizes objective and rapid diagnosis, product recommendation, and first aid guidance by AI, thereby achieving remarkable effects such as improved survival rate, reduced risk of misdiagnosis, optimized product selection, and reduced user burden. Specific application fields include self-medication support in drugstore stores, initial response in emergency situations, family support in home care settings, and emergency response at event venues or on public transportation.

Step 1: The acquisition unit acquires an image of the emergency patient. The image of the emergency patient includes still images, videos, X-ray images, and the like. The acquisition unit uses cameras built into AR glasses, smartphones, or tablets to acquire images of the emergency patient in real time. For example, when the emergency patient has collapsed, the posture and facial expression are photographed in detail. Step 2: The analysis unit analyzes the image acquired by the acquisition unit using generative AI. The analysis is performed based on image processing algorithms and diagnostic criteria. The generative AI analyzes the facial color, facial expression, posture, etc., of the emergency patient and diagnoses the condition. The generative AI can also analyze the image of the emergency patient using a text-generating AI (e.g., LLM) or a multimodal generative AI. Step 3: The proposal unit proposes a product based on the diagnosis result obtained by the analysis unit. The proposed products include beverages for hydration, antipyretics, analgesics, and the like. The proposal unit uses generative AI to propose products based on the diagnosis result. For example, when the emergency patient is experiencing dehydration, a beverage for hydration is proposed; when the emergency patient shows symptoms such as headache or fever, antipyretics or analgesics are proposed. The proposal unit has a function to notify the user of the proposed product and displays product information on the display of AR glasses, smartphones, or tablets. Specifically, in Step 1, the acquisition unit uses AR glasses, smartphones, or tablet devices equipped with high-resolution camera modules (e.g., 1920×1080 pixels, 30 fps support) to acquire real-time images of the entire body of the emergency patient, close-up images of the face, and continuous images from multiple angles (video streams). The acquisition unit transmits the image data to a preprocessing unit, which performs noise removal (e.g., Gaussian filter), contrast adjustment, and segmentation of face and limb regions (e.g., using object detection models such as YOLOv8 or U-Net). The preprocessed images are numerically converted by a feature extraction unit into multidimensional feature vectors (e.g., 512 dimensions) such as facial color (RGB histogram, complexion estimation), facial expression (facial muscle patterns, action units), and posture (landmark extraction by skeleton estimation algorithms). In Step 2, the analysis unit inputs these feature vectors into a multimodal neural network (e.g., a model combining a ResNet-50 image input unit and a Transformer-based text encoder) and outputs the condition of the emergency patient (e.g., dehydration, fever, impaired consciousness, trauma, etc.) as a probability distribution (e.g., a score of 0.0 to 1.0 for each symptom). For example, image tensors of “pale facial color, agonized expression, supine posture” and speech recognition text such as “collapsed,” “unconscious,” etc., can be input simultaneously to the AI. Example AI outputs include “dehydration: 0.85,” “fever: 0.10,” “trauma: 0.05.” The analysis unit determines these scores using a threshold determination unit and performs rule-based processing such as “if the dehydration score is 0.7 or higher, recommend a beverage for hydration,” “if the fever score is 0.5 or higher, recommend an antipyretic,” etc. In Step 3, the proposal unit matches the diagnosis result with the drugstore product database (structured data such as product name, ingredients, indications, inventory information, etc.) and generates an optimal product candidate list (e.g., oral rehydration solution, antipyretic analgesics, cooling sheets, etc.). The product proposal unit inputs the diagnosis result and user attributes (age, gender, medical history, etc.) into a generative AI (e.g., LLM-based recommendation model) and can generate recommendation sentences with explanations such as “Recommended product: oral rehydration solution, reason: strong dehydration symptoms, rapid hydration required.” Example outputs include “Recommended product: oral rehydration solution, recommended reason: strong suspicion of dehydration based on facial color and posture” and “Recommended product: antipyretic, recommended reason: high fever score.” These recommendation results are overlay-displayed on the AR glasses display, and the user can select products or display additional information via voice commands or touchpad operations. As a technical effect, the system, unlike conventional responses dependent on human visual judgment or empirical rules, integrally analyzes diverse data such as images, audio, and text in a high-dimensional space and realizes objective and rapid diagnosis, product recommendation, and first aid guidance by AI, thereby achieving remarkable effects such as improved survival rate, reduced risk of misdiagnosis, optimized product selection, and reduced user burden. Specific application fields include self-medication support in drugstore stores, initial response in emergency situations, family support in home care settings, and emergency response at event venues or on public transportation.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned acquisition unit, analysis unit, proposal unit, and display unit is implemented, for example, by at least one of a smart device 14 and a data processing apparatus 12. For example, the acquisition unit acquires an image of an emergency patient in real time using a camera 42 of the smart device 14. The analysis unit is implemented by a specific processing unit 290 of the data processing apparatus 12 and analyzes the image of the emergency patient using generative AI. The proposal unit is implemented by the specific processing unit 290 of the data processing apparatus 12 and proposes a product based on the diagnosis result. The display unit displays procedures for first aid on a display 40A of the smart device 14. The correspondence between each unit and the device or control unit is not limited to the examples described above and various modifications are possible.

### Second Embodiment

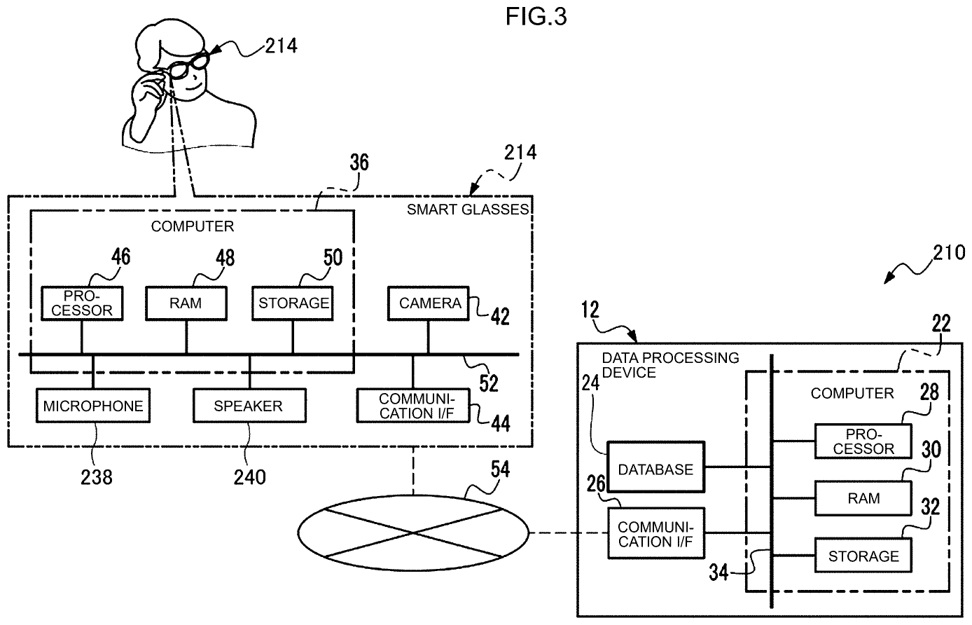

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

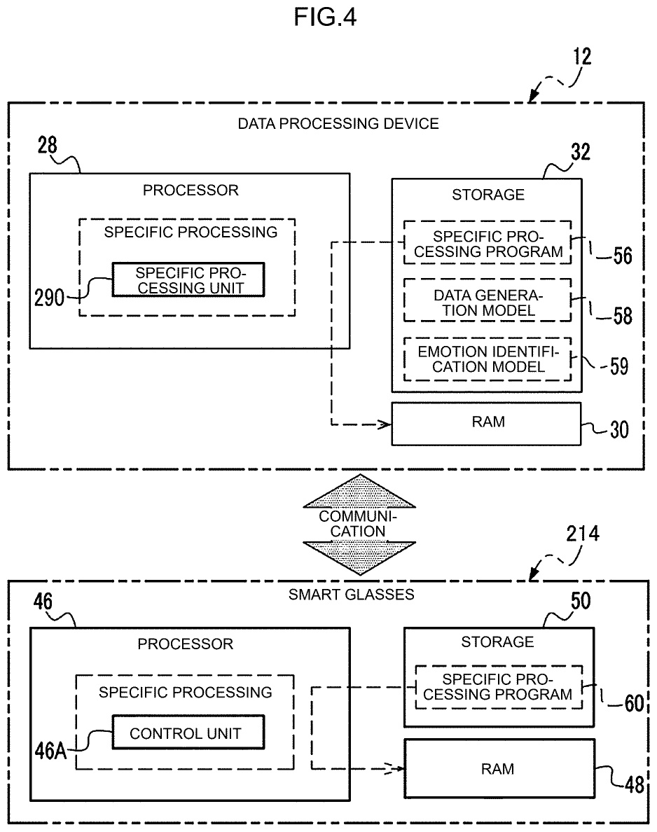

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned acquisition unit, analysis unit, proposal unit, and display unit is implemented, for example, by at least one of smart glasses 214 and a data processing apparatus 12. For example, the acquisition unit acquires an image of an emergency patient in real time using a camera 42 of the smart glasses 214. The analysis unit is implemented by a specific processing unit 290 of the data processing apparatus 12 and analyzes the image of the emergency patient using generative AI. The proposal unit is implemented by the specific processing unit 290 of the data processing apparatus 12 and proposes a product based on the diagnosis result. The display unit displays procedures for first aid on a display of the smart glasses 214. The correspondence between each unit and the device or control unit is not limited to the examples described above and various modifications are possible.

### Third Embodiment

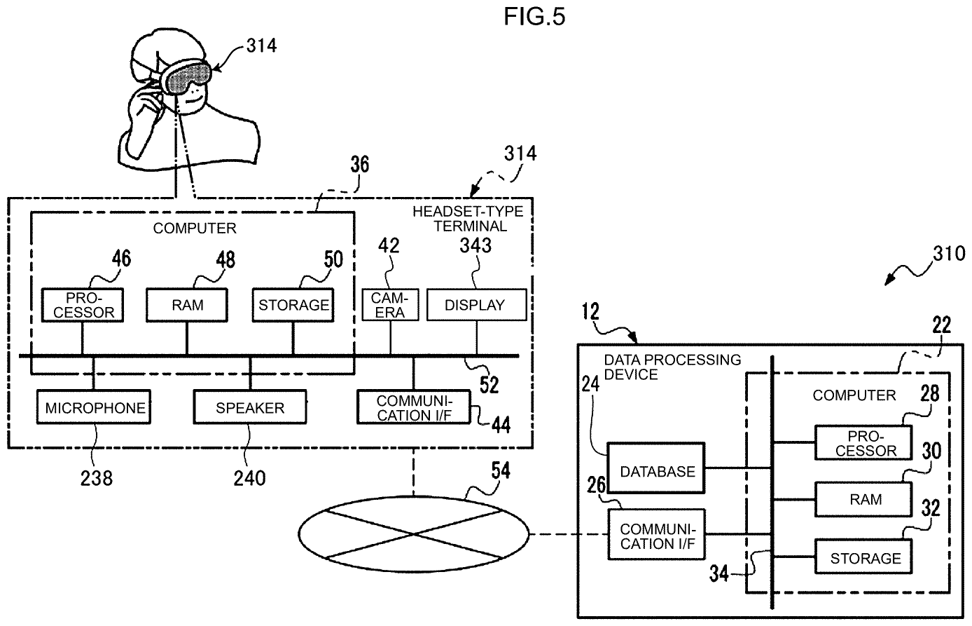

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

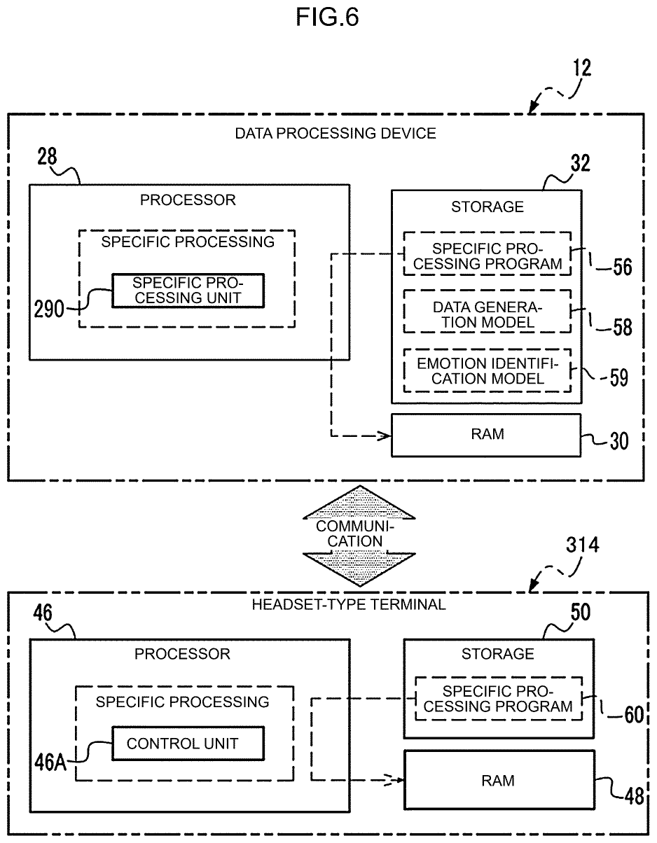

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned acquisition unit, analysis unit, proposal unit, and display unit is implemented, for example, by at least one of a headset-type terminal 314 and a data processing apparatus 12. For example, the acquisition unit acquires an image of an emergency patient in real time using a camera 42 of the headset-type terminal 314. The analysis unit is implemented by a specific processing unit 290 of the data processing apparatus 12 and analyzes the image of the emergency patient using generative AI. The proposal unit is implemented by the specific processing unit 290 of the data processing apparatus 12 and proposes a product based on the diagnosis result. The display unit displays procedures for first aid on a display 343 of the headset-type terminal 314. The correspondence between each unit and the device or control unit is not limited to the examples described above and various modifications are possible.

### Fourth Embodiment

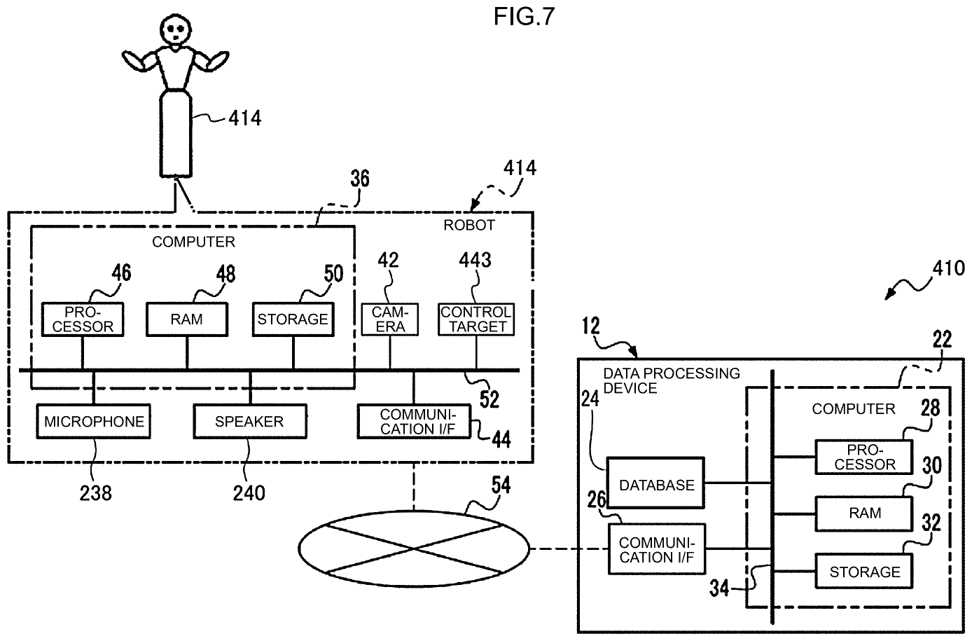

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

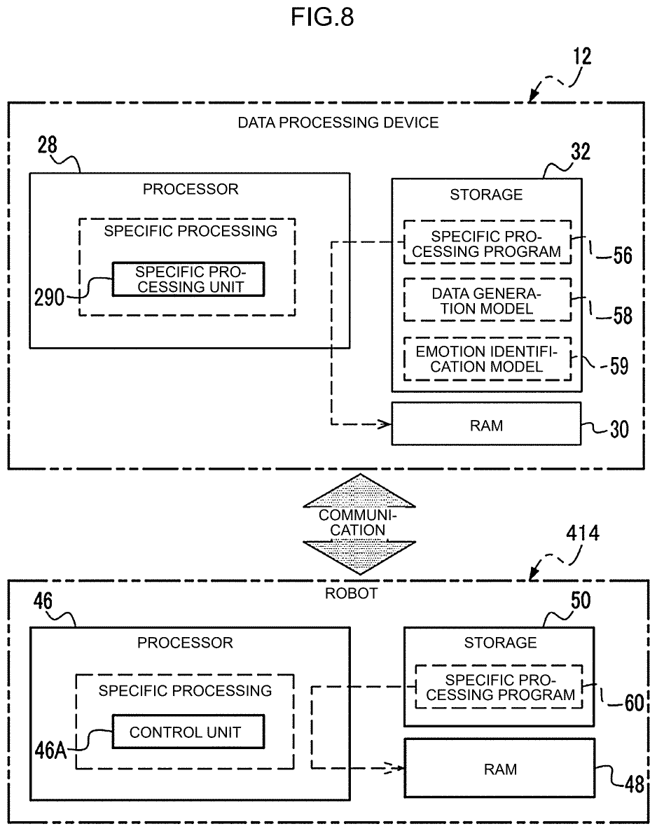

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned acquisition unit, analysis unit, proposal unit, and display unit is implemented, for example, by at least one of a robot 414 and a data processing apparatus 12. For example, the acquisition unit acquires an image of an emergency patient in real time using a camera 42 of the robot 414. The analysis unit is implemented by a specific processing unit 290 of the data processing apparatus 12 and analyzes the image of the emergency patient using generative AI. The proposal unit is implemented by the specific processing unit 290 of the data processing apparatus 12 and proposes a product based on the diagnosis result. The display unit displays procedures for first aid on a display of the robot 414. The correspondence between each unit and the device or control unit is not limited to the examples described above and various modifications are possible.

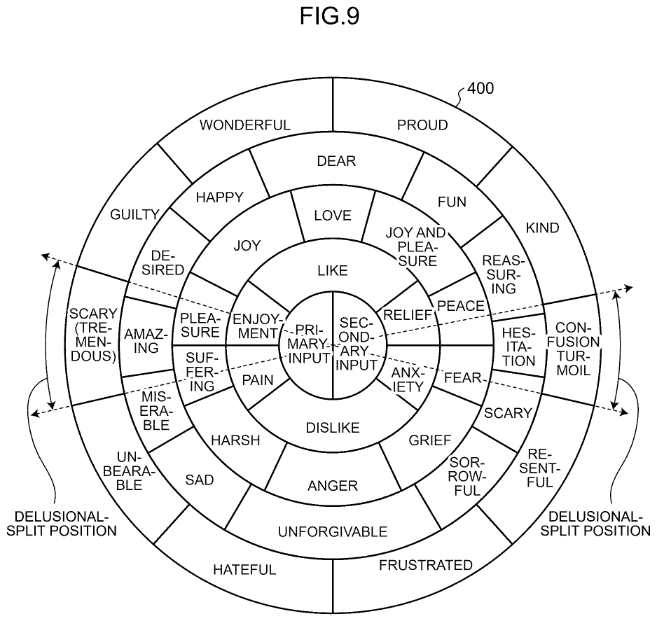

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

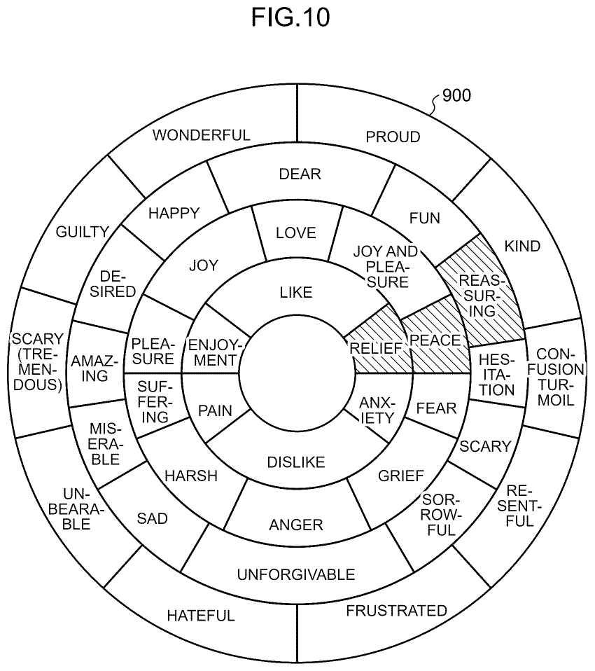

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1) A system comprising: an acquisition unit configured to acquire an image of an emergency patient; an analysis unit configured to analyze the image acquired by the acquisition unit; and a proposal unit configured to propose a product based on a diagnosis result obtained by the analysis unit.
(Supplementary Note 2) The system according to Supplementary Note 1, wherein the proposal unit is configured to propose a product based on the diagnosis result by means of generative AI.
(Supplementary Note 3) The system according to Supplementary Note 1, wherein the proposal unit is configured to propose a product corresponding to the condition of the emergency patient by means of generative AI.
(Supplementary Note 4) The system according to Supplementary Note 1, further comprising a display unit configured to display procedures for first aid.
(Supplementary Note 5) The system according to Supplementary Note 3, wherein the display unit is configured to display procedures for first aid for cardiopulmonary resuscitation or hemostasis.
(Supplementary Note 6) The system according to Supplementary Note 1, wherein the acquisition unit is configured to estimate a user's emotion and adjust the timing of acquiring the image of the emergency patient based on the estimated emotion of the user.
(Supplementary Note 7) The system according to Supplementary Note 1, wherein the acquisition unit is configured to analyze a user's past emergency patient response history and select an image acquisition method.
(Supplementary Note 8) The system according to Supplementary Note 1, wherein the acquisition unit is configured to perform filtering based on the user's current location information or environment when acquiring an image of the emergency patient.
(Supplementary Note 9) The system according to Supplementary Note 1, wherein the acquisition unit is configured to estimate a user's emotion and determine a priority order of images to be acquired based on the estimated emotion of the user.
(Supplementary Note 10) The system according to Supplementary Note 1, wherein the acquisition unit is configured to analyze audio information around the user when acquiring an image of the emergency patient and preferentially acquire related images.
(Supplementary Note 11) The system according to Supplementary Note 1, wherein the acquisition unit is configured to analyze a user's online activity when acquiring an image of the emergency patient and acquire related images.
(Supplementary Note 12) The system according to Supplementary Note 1, wherein the analysis unit is configured to estimate a user's emotion and adjust the accuracy of image analysis based on the estimated emotion of the user.
(Supplementary Note 13) The system according to Supplementary Note 1, wherein the analysis unit is configured to refer to past health data of the emergency patient during image analysis to improve the accuracy of the analysis.
(Supplementary Note 14) The system according to Supplementary Note 1, wherein the analysis unit is configured to perform analysis based on current environment information of the emergency patient during image analysis.
(Supplementary Note 15) The system according to Supplementary Note 1, wherein the analysis unit is configured to estimate a user's emotion and adjust a display method of analysis results based on the estimated emotion of the user.
(Supplementary Note 16) The system according to Supplementary Note 1, wherein the analysis unit is configured to perform analysis based on a geographical distribution of emergency patients during image analysis.
(Supplementary Note 17) The system according to Supplementary Note 1, wherein the analysis unit is configured to refer to related literature of the emergency patient during image analysis to improve the accuracy of the analysis.
(Supplementary Note 18) The system according to Supplementary Note 1, wherein the proposal unit is configured to estimate a user's emotion and adjust a method of expressing product proposals based on the estimated emotion of the user.
(Supplementary Note 19) The system according to Supplementary Note 1, wherein the proposal unit is configured to adjust a level of detail of proposals based on the condition of the emergency patient when proposing a product.
(Supplementary Note 20) The system according to Supplementary Note 1, wherein the proposal unit is configured to apply different proposal algorithms according to a category of the emergency patient when proposing a product.
(Supplementary Note 21) The system according to Supplementary Note 1, wherein the proposal unit is configured to estimate a user's emotion and adjust a length of proposals based on the estimated emotion of the user.
(Supplementary Note 22) The system according to Supplementary Note 1, wherein the proposal unit is configured to determine a priority order of proposals based on the condition of the emergency patient when proposing a product.
(Supplementary Note 23) The system according to Supplementary Note 1, wherein the proposal unit is configured to adjust an order of proposals based on relevance to the emergency patient when proposing a product.
(Supplementary Note 24) The system according to Supplementary Note 3, wherein the display unit is configured to estimate a user's emotion and adjust a display method of procedures for first aid based on the estimated emotion of the user.
(Supplementary Note 25) The system according to Supplementary Note 3, wherein the display unit is configured to refer to a user's past first aid history when displaying procedures for first aid and select an optimal display method.
(Supplementary Note 26) The system according to Supplementary Note 3, wherein the display unit is configured to customize display content based on the current condition of the emergency patient when displaying procedures for first aid.
(Supplementary Note 27) The system according to Supplementary Note 3, wherein the display unit is configured to estimate a user's emotion and adjust a display order of procedures for first aid based on the estimated emotion of the user.
(Supplementary Note 28) The system according to Supplementary Note 3, wherein the display unit is configured to consider a user's device information when displaying procedures for first aid and select an optimal display method.
(Supplementary Note 29) The system according to Supplementary Note 3, wherein the display unit is configured to refer to related literature of the emergency patient when displaying procedures for first aid to improve display content.

What is claimed is:

1. A system comprising:
circuitry configured to:
acquire image data from an imaging device communicatively coupled to the system via a packet-switched network;
generate, by inputting the image data into a trained neural network, a multidimensional feature vector representing attributes extracted from the image data;
generate, by inputting the multidimensional feature vector into a data generation model, inference data indicating a classification result derived from the multidimensional feature vector; and
transmit, to a client terminal communicatively coupled to the system via the packet-switched network, output data generated based on the inference data.

2. The system according to claim 1, wherein the image data comprises image data of a subject captured by a camera of the imaging device, and the multidimensional feature vector represents at least one of facial color, expression, or posture attributes of the subject.

3. The system according to claim 2, wherein the trained neural network comprises a multimodal neural network configured to receive the image data and text data simultaneously, and the multimodal neural network comprises a convolutional neural network for the image data and a Transformer-based encoder for the text data.

4. The system according to claim 1, wherein the inference data indicates a condition of an emergency patient as a probability distribution comprising a score of 0.0 to 1.0 for each of a plurality of symptom categories.

5. The system according to claim 4, wherein the output data comprises a recommendation of a product corresponding to the condition of the emergency patient, and the data generation model comprises a large language model configured to generate the recommendation based on the inference data.

6. The system according to claim 1, wherein the circuitry is further configured to generate, by inputting biometric sensor data and the image data into an emotion identification model, an emotion value indicating an estimated emotion of a user of the imaging device.

7. The system according to claim 6, wherein the emotion identification model comprises a multimodal neural network configured to receive a facial expression image, audio data, and biometric sensor data, and to output emotion labels as a probability distribution.

8. The system according to claim 6, wherein the circuitry is further configured to adjust a timing of acquiring the image data based on the emotion value.

9. The system according to claim 6, wherein the circuitry is further configured to adjust an accuracy parameter of the trained neural network based on the emotion value, the accuracy parameter comprising at least one of a threshold for object detection, a number of ensemble inferences, or a strength of image preprocessing.

10. The system according to claim 6, wherein the circuitry is further configured to adjust a length or a level of detail of the output data based on the emotion value.

11. The system according to claim 1, wherein the circuitry is further configured to:
acquire audio data from a microphone of the imaging device;
generate, by inputting the audio data into an audio event detection neural network, audio event scores indicating a probability of each of a plurality of audio event categories; and
determine, based on the audio event scores, a priority order for acquiring additional image data.

12. The system according to claim 11, wherein the audio event detection neural network comprises a hybrid model of a convolutional neural network and a recurrent neural network, and the audio data is converted into feature vectors using mel-frequency cepstral coefficient extraction.

13. The system according to claim 1, wherein the circuitry is further configured to:
retrieve, from a database, historical data associated with a subject depicted in the image data; and
generate the inference data by inputting both the multidimensional feature vector and the historical data into the data generation model.

14. The system according to claim 1, wherein the circuitry is further configured to:
acquire environment data comprising at least one of ambient temperature, humidity, illuminance, or noise level from an environmental sensor; and
adjust a parameter of the trained neural network based on the environment data.

15. The system according to claim 1, wherein the circuitry is further configured to:
determine, based on the inference data, that a procedural guide is to be presented; and
transmit, to the client terminal, procedural guide data extracted from a procedure database, the procedural guide data comprising step-by-step instructions in at least one of text, image, video, or audio format.

16. The system according to claim 15, wherein the circuitry is further configured to adjust a display order of the step-by-step instructions based on an emotion value estimated from sensor data of a user of the client terminal.

17. The system according to claim 1, wherein the circuitry is further configured to:
determine a category of a subject depicted in the image data based on attribute data comprising at least one of age, gender, or symptom category; and
select, based on the determined category, a proposal algorithm from a plurality of proposal algorithms, each proposal algorithm corresponding to a different category.

18. A system comprising:
a communication interface comprising a communication processor and an antenna, the communication interface configured to manage communication via a packet-switched network conforming to at least one of a 5G, Wi-Fi, or Bluetooth communication standard;
a processor comprising at least one of a CPU, a GPU, or a TPU;
a random-access memory connected to the processor via a bus;
a non-volatile storage device storing a data generation model obtained by performing deep learning on a neural network and an emotion identification model;
a database connected to the bus; and
circuitry configured to:
receive, via the communication interface, image data captured by a CMOS image sensor or a CCD image sensor of a client terminal communicatively coupled to the system via the packet-switched network;
generate, by inputting the image data into a multimodal neural network comprising a ResNet-50 convolutional neural network for image input and a Transformer-based encoder for text input, a multidimensional feature vector of at least 512 dimensions representing attributes extracted from the image data;
generate, by inputting the multidimensional feature vector into the data generation model, inference data indicating a classification result as a probability distribution;
generate, by inputting the inference data and attribute data into the data generation model comprising a large language model, output data comprising a structured recommendation; and
transmit, via the communication interface, the output data to the client terminal for presentation on a display of the client terminal.

19. The system according to claim 18, wherein the circuitry is further configured to:
receive, via the communication interface, audio data captured by a microphone of the client terminal;
generate, by inputting the audio data into an audio event detection model comprising a CNN and RNN hybrid architecture, audio event scores; and
determine, based on the audio event scores, a priority order for acquiring additional image data from the client terminal.

20. A method performed by circuitry of a system, the method comprising:
acquiring image data from an imaging device communicatively coupled to the system via a packet-switched network;
generating, by inputting the image data into a trained neural network, a multidimensional feature vector representing attributes extracted from the image data;
generating, by inputting the multidimensional feature vector into a data generation model, inference data indicating a classification result derived from the multidimensional feature vector; and
transmitting, to a client terminal communicatively coupled to the system via the packet-switched network, output data generated based on the inference data.
